SEQUENCE LISTING (25 sequences)

SEQ ID NO: 1 - DNA, 1308 bp, Bacillus sp.
  CDS (1)..(1305)
atgagcggttttacagcgttaagtgaagcagaattaaatgacctatatgcagcacgacaaaaagagtatgaaacgtacaaaagtaaaaacttacacttagacatgtctagagggaaaccttcaccaaaacagctcgatttatctatgggcatgctcgatgtcgtgacatcaaaggatgcaatgacagctgaggatggtacagatgtgcgaaactatggcggcttgacaggccttcctgaaacaaagaaattttttgcaagtgtgctcaatctgaagccagaacaaatcatcattggcggtaattctagcctaaatatgatgcatgacacaattgcccgtgctatgactcacggcgtatatggcagcaaaacaccttggggagagcttccaaaggtaaaattccttgcaccaagcccagggtatgatcgtcattttgccatttgtgagcattttaacatagagatgattacggtagatatgaagtcggatggacctgacatggatcaggtggaaaaattggttgcagaagatgaagccatcaaagggatttggtgtgtaccaaaatatagcaaccctgacggcattacgtattcagatgaggttgtcgaccgtcttgcttccatgcagacaaaagcagacgacttccgtattttttgggatgatgcctatgcagtccaccatctaacagatacgcctgatacgttaaaagatatttttcaagcagtagacaaagcagggcatgcaaaccgtgtgtttatgttcgcctctacttctaaaattacgttcccaggctcaggtgttgcactgatggcatctagtcaggacaacgtcagctttattcaaaaacagctatcagttcaaaccattgggccagataaaatcaatcaattaagacaccttcgtttcttcaagaatccagaaggattgaaggaacatatgaaaaagcatgcagcgattattaagccgaaatttgacctcgttctttcgatccttgatgaaaagcttggtggaacaggcatcgctgagtggcacaaaccaaatggcggatattttattagcttaaatacactcgatcattgtgcaaaagctgttgtgcaaaaagcgaaagaagccggtgtgacactaacaggtgcaggggcgacatatccttatggaaacgacccgcttgatcgtaacatccgtattgcgccaacgttcccaacgcttgaagaactagagcaggcgattgatatctttacgttatgcgttcagcttgtcagcattgaaaagctgctgtctgagaaaagtcaatcagcaccaacggtataa

SEQ ID NO: 2 - protein, 435 aa, Bacillus sp.
MSGFTALSEAELNDLYAARQKEYETYKSKNLHLDMSRGKPSPKQLDLSMGMLDVVTSKDAMTAEDGTDVRNYGGLTGLPETKKFFASVLNLKPEQIIIGGNSSLNMMHDTIARAMTHGVYGSKTPWGELPKVKFLAPSPGYDRHFAICEHFNIEMITVDMKSDGPDMDQVEKLVAEDEAIKGIWCVPKYSNPDGITYSDEVVDRLASMQTKADDFRIFWDDAYAVHHLTDTPDTLKDIFQAVDKAGHANRVFMFASTSKITFPGSGVALMASSQDNVSFIQKQLSVQTIGPDKINQLRHLRFFKNPEGLKEHMKKHAAIIKPKFDLVLSILDEKLGGTGIAEWHKPNGGYFISLNTLDHCAKAVVQKAKEAGVTLTGAGATYPYGNDPLDRNIRIAPTFPTLEELEQAIDIFTLCVQLVSIEKLLSEKSQSAPTV

SEQ ID NO: 3 - DNA, 1678 bp, Bacillus sp.
gaaacaattactcaaagaagcccgcgatatattcccgctgactgaagctgcacatgatttcttttcttttcctattgaccgtaccctgtaaaaattggatgaatcgtcaaaaatagttgtgtgatttttttgatatttatgatgacgctctttttcaggtagtggtaaaatggtgaagaaaaaacaaatgacgaattacactatgaagaatacgggaggcacaatcaaagatgagcggttttacagcgttaagtgaagcagaattaaatgacctatatgcagcacgacaaaaagagtatgaaacgtacaaaagtaaaaacttacacttagacatgtctagagggaaaccttcaccaaaacagctcgatttatctatgggcatgctcgatgtcgtgacatcaaaggatgcaatgacagctgaggatggtacagatgtgcgaaactatggcggcttgacaggccttcctgaaacaaagaaattttttgcaagtgtgctcaatctgaagccagaacaaatcatcattggcggtaattctagcctaaatatgatgcatgacacaattgcccgtgctatgactcacggcgtatatggcagcaaaacaccttggggagagcttccaaaggtaaaattccttgcaccaagcccagggtatgatcgtcattttgccatttgtgagcattttaacatagagatgattacggtagatatgaagtcggatggacctgacatggatcaggtggaaaaattggttgcagaagatgaagccatcaaagggatttggtgtgtaccaaaatatagcaaccctgacggcattacgtattcagatgaggttgtcgaccgtcttgcttccatgcagacaaaagcagacgacttccgtattttttgggatgatgcctatgcagtccaccatctaacagatacgcctgatacgttaaaagatatttttcaagcagtagacaaagcagggcatgcaaaccgtgtgtttatgttcgcctctacttctaaaattacgttcccaggctcaggtgttgcactgatggcatctagtcaggacaacgtcagctttattcaaaaacagctatcagttcaaaccattgggccagataaaatcaatcaattaagacaccttcgtttcttcaagaatccagaaggattgaaggaacatatgaaaaagcatgcagcgattattaagccgaaatttgacctcgttctttcgatccttgatgaaaagcttggtggaacaggcatcgctgagtggcacaaaccaaatggcggatattttattagcttaaatacactcgatcattgtgcaaaagctgttgtgcaaaaagcgaaagaagccggtgtgacactaacaggtgcaggggcgacatatccttatggaaacgacccgcttgatcgtaacatccgtattgcgccaacgttcccaacgcttgaagaactagagcaggcgattgatatctttacgttatgcgttcagcttgtcagcattgaaaagctgctgtctgagaaaagtcaatcagcaccaacggtataacgaaaaaactccttgactgatgtccggtcaaggagtttttgtttttagttagctgtttgataataagtggcaggctgttttgctgcacaccatacatcataaatcgcaagctgcacaatatgtggttcatcgtgatgaat

SEQ ID NO: 4 - protein, 15 aa, Bacillus sp.
SGFTALSEAELNDLY

SEQ ID NO: 5 - protein, 12 aa, Bacillus sp.
QLDLSMGMLDVV

SEQ ID NO: 6 - DNA, 30 bp, Artificial Sequence
  Description of Artificial Sequence Synthetic forward primer for amplifying DNA fragment containing aminotransferase gene derived from Bacillus sp. (Bp-u200-f)
ctcaggaagcaggcgcaaaaagattaattt

SEQ ID NO: 7 - DNA, 30 bp, Artificial Sequence
  Description of Artificial Sequence Synthetic reverse primer for amplifying DNA fragment containing  aminotransferase gene derived from Bacillus sp. (Bp-d200-r)
ggatgctgtctttgtcatcccaaagtggat

SEQ ID NO: 8 - DNA, 30 bp, Artificial Sequence
  Description of Artificial Sequence Synthetic forward primer for amplifying DNA fragment containing aminotransferase gene derived from Bacillus sp. (1616AT-Nde-f)
ggaattccatatgagcggttttacagcgtt

SEQ ID NO: 9 - DNA, 40 bp, Artificial Sequence
  Description of Artificial Sequence Synthetic reverse primer for amplifying DNA fragment containing aminotransferase gene derived from Bacillus sp. (1616-xho-r)
gtcaaggagtttttctcgagtaccgttggtgctgattgac

SEQ ID NO: 10 - DNA, 29 bp, Artificial Sequence
  Description of Artificial Sequence Synthetic forward primer for converting DNA sequence recognized by NdeI, which is found on aminotransferase gene derived from Bacillus sp. (1616-delNde-f)
ggattgaaggaacacatgaaaaagcatgc

SEQ ID NO: 11 - DNA, 29 bp, Artificial Sequence
  Description of Artificial Sequence Synthetic reverse primer for converting DNA sequence recognized by NdeI, which is found on aminotransferase gene derived from Bacillus sp. (1616-delNde-r)
gcatgctttttcatgtgttccttcaatcc

SEQ ID NO: 12 - DNA, 30 bp, Artificial Sequence
  Description of Artificial Sequence Synthetic forward primer for amplifying DNA fragment containing SpAld gene (SpAld-f-NdeI)
ggaattccatatgacccagacgcgcctcaa

SEQ ID NO: 13 - DNA, 30 bp, Artificial Sequence
  Description of Artificial Sequence Synthetic reverse primer for amplifying DNA fragment containing SpAld gene (SpAld-r-HindIII)
gcccaagctttcagtaccccgccagttcgc

SEQ ID NO: 14 - DNA, 29 bp, Artificial Sequence
  Description of Artificial Sequence Synthetic forward primer for converting rare codon 6L in SpAld gene (6L-f)
acccagacgcgcctgaacggcatcatccg

SEQ ID NO: 15 - DNA, 29 bp, Artificial Sequence
  Description of Artificial Sequence Synthetic reverse primer for converting rare codon 6L in SpAld gene (6L-r)
cggatgatgccgttcaggcgcgtctgggt

SEQ ID NO: 16 - DNA, 29 bp, Artificial Sequence
  Description of Artificial Sequence Synthetic forward primer for converting rare codon 13L in SpAld gene (13L-f)
atcatccgcgctctggaagccggcaagcc

SEQ ID NO: 17 - DNA, 29 bp, Artificial Sequence
  Description of Artificial Sequence Synthetic reverse primer for converting rare codon 13L in SpAld gene (13L-r)
ggcttgccggcttccagagcgcggatgat

SEQ ID NO: 18 - DNA, 29 bp, Artificial Sequence
  Description of Artificial Sequence Synthetic forward primer for converting rare codon 18P in SpAld gene (18P-f)
gaagccggcaagccggctttcacctgctt

SEQ ID NO: 19 - DNA, 29 bp, Artificial Sequence
  Description of Artificial Sequence Synthetic reverse primer for converting rare codon 18P in SpAld gene (18P-r)
aagcaggtgaaagccggcttgccggcttc

SEQ ID NO: 20 - DNA, 29 bp, Artificial Sequence
  Description of Artificial Sequence Synthetic forward primer for converting rare codon 38P in SpAld gene (38P-f)
ctgaccgatgccccgtatgacggcgtggt

SEQ ID NO: 21 - DNA, 29 bp, Artificial Sequence
  Description of Artificial Sequence Synthetic reverse primer for converting rare codon 38P in SpAld gene (38P-r)
accacgccgtcatacggggcatcggtcag

SEQ ID NO: 22 - DNA, 29 bp, Artificial Sequence
  Description of Artificial Sequence Synthetic forward primer for converting rare codon 50P in SpAld gene (50P-f)
atggagcacaacccgtacgatgtcgcggc

SEQ ID NO: 23 - DNA, 29 bp, Artificial Sequence
  Description of Artificial Sequence Synthetic reverse primer for converting rare codon 50P in SpAld gene (50P-r)
gccgcgacatcgtacgggttgtgctccat

SEQ ID NO: 24 - DNA, 42 bp, Artificial Sequence
  Description of Artificial Sequence Synthetic forward primer for converting rare codons 77P, 81P and 84R in SpAld gene (77P-81P-84R-f)
cggtcgcgccgtcggtcaccccgatcgcgcgcatcccggcca

SEQ ID NO: 25 - DNA, 42 bp, Artificial Sequence
  Description of Artificial Sequence Synthetic reverse primer for converting rare codons 77P, 81P and 84R in SpAld gene (77P-81P-84R-r)
tggccgggatgcgcgcgatcggggtgaccgacggcgcgaccg

(19) United States
(12) Patent Application Publication
(10) Pub. No.: US 2012/0208244 A1
(43) Pub. Date: Aug. 16, 2012

(54) METHOD FOR PRODUCING MONATIN

(75) Inventors: YASUAKI TAKAKURA, Kanagawa (JP); HIROOMI OGINO, Kanagawa (JP)

(73) Assignee: AJINOMOTO CO., INC., TOKYO (JP)

(21) Appl. No.: 13/189,799

(22) Filed: Jul. 25, 2011

Related U.S. Application Data
(60) Provisional application No. 61/393,632, filed on Oct. 15, 2010.

(51) Int. Cl. C12P 13/04; C12N 15/63; C12N 15/54; C12N 1/19; C12N 1/15; C12N 9/10; C12N 1/21

(52) U.S. Cl. 435/106; 435/193; 435/320.1; 435/252.33; 435/252.31; 435/254.21; 435/254.23; 435/254.3; 536/23.2; 435/252.32

(57) ABSTRACT

[Problem] Providing a methodology for improving a yield of 2R,4R-Monatin.

[Means for solving the Problem] A method for producing 2S,4R-Monatin or a salt thereof, comprising contacting 4R-IHOG with an L-aminotransferase in the presence of an L-amino acid to form the 2S,4R-Monatin; a method for producing 2R,4R-Monatin or a salt thereof, comprising isomerizing the 2S,4R-Monatin to form the 2R,4R-Monatin; and the like. These production methods may further comprise condensing indole-3-pyruvate and pyruvate to form the 4R-IHOG, and oxidizing a tryptophan to form the indole-3-pyruvate.

### TECHNICAL FIELD

The present invention relates to a method for producing Monatin using an L-aminotransferase, and the like.

### BACKGROUND ART

Monatin [4-(indole-3-yl-methyl)-4-hydroxy-glutamic acid] is a compound that is one of amino acids contained in roots of Schlerochitom ilicifolius that is a shrub in South Africa and is particularly expected as a low calorie sweetener because of having sweetness one thousand and several hundreds times sweeter than sucrose (see Patent Document 1). The Monatin has asymmetric carbon atoms at positions 2 and 4, and a naturally occurring stereoisomer of Monatin is a 2S, 4S-isomer. Naturally non-occurring three stereoisomers have been synthesized by organic chemistry processes. All of these stereoisomers are excellent in sweetness, and expected to be used as the sweeteners.

Several methods have been reported as the methods for producing the Monatin (e.g., see Patent Document 2). However, all of the reported methods require a step of multiple stages, and thus, it is required to improve a synthetic yield of the Monatin.

Specifically, for the method for producing the Monatin, the following method for producing 2R,4R-Monatin by synthesizing indole-3-pyruvate (hereinafter referred to as “IPA” as needed) from L-tryptophan (L-Trp), synthesizing 4-(indole-3-yl-methyl)-4-hydroxy-2-oxoglutaric acid (hereinafter referred to as “4R-IHOG” as needed) that is a 4R-isomer from the resulting IPA and pyruvate, and subsequently subjecting the obtained 4R-IHOG to an oximation reaction, a reduction reaction and an epimerization-crystallization method has been known (conventional method (1)) (see Patent Document 2).

However, an aldolase step (second step) is an equilibrium reaction, and thus, a satisfactory yield is not always obtained in this reaction.

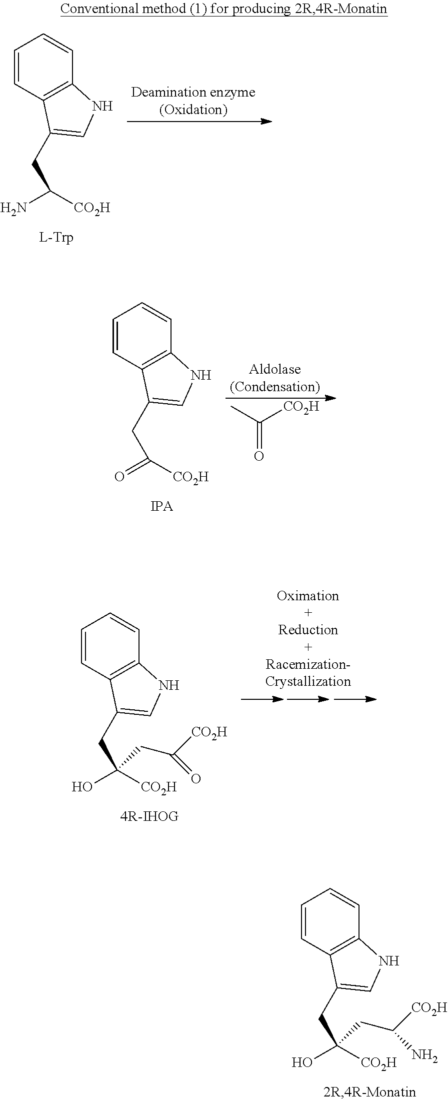

In order to improve the yield of the 2R,4R-Monatin, the method for producing the 2R,4R-Monatin by a one-pot enzymatic reaction has been invented (conventional method (2)) (see Patent Documents 3 to 6).
Patent Document 1: JP Sho-64-25757-A
Patent Document 2: International Publication WO2003/059865
Patent Document 3: International Publication WO2007/133184
Patent Document 4: International Publication WO2005/042756
Patent Document 5: US Patent Application Publication No. 2006/0252135 Specification
Patent Document 6: US Patent Application Publication No. 2008/020434 Specification

### SUMMARY OF INVENTION

### Problem to be Solved by the Invention

The object of the present invention is to provide a method for producing Monatin with a good yield.

### Means for Solving Problem

As a result of an extensive study, the present inventors have found that the above problem can be solved by using an L-aminotransferase, and completed the present invention. No L-aminotransferase that acts upon 4R-IHOG has been known so far.

Accordingly, the present invention is as follows.

[1] A method for producing 2S,4R-Monatin or a salt thereof, comprising contacting 4R-IHOG with an L-aminotransferase in the presence of an L-amino acid to form the 2S,4R-Monatin.
[2] The production method of [1], further comprising contacting an oxy derivative of the L-amino acid with a decarboxylase to degrade the oxy derivative of the L-amino acid, wherein the oxy derivative of the L-amino acid is formed from the L-amino acid due to action of the L-aminotransferase.
[3] The production method of [1] or [2], wherein the L-amino acid is L-aspartate.
[4] The production method of [3], further comprising contacting oxaloacetate with an oxaloacetate decarboxylase to irreversibly form pyruvate, wherein the oxaloacetate is formed from the L-aspartate by action of the L-aminotransferase.
[5] The production method of any of [1]-[4], wherein the L-aminotransferase is derived from a microorganism belonging to genus Achromobacter, genus Alcaligenes, genus Arthrobacter, genus Bacillus, genus Candida, genus Corynebacterium, genus Lodderomyce, genus Micrococcus, genus Microbacterium, genus Nocardia, genus Pseudomonas, genus Rhizobium, genus Stenotrophomonas, genus Xanthomonas, or genus Yarrowia.
[6] The production method of [5], wherein the L-aminotransferase is derived from a microorganism belonging to Achromobacter brunificans, Achromobacter butyri, Alcaligenes faecalis, Alcaligenes metalcaligenes, Arthrobacter ureafaciens, Bacillus sp., Candida norvegensis, Candida inconspicua, Corynebacterium ammoniagenes, Lodderomyces elongisporus, Micrococcus luteus, Microbacterium sp., Nocardia globerula, Pseudomonas betainovorans, Pseudomonas chlororaphis, Pseudomonas citronocllolis, Pseudomonas fragi, Pseudomonas hydrogenovora, Pseudomonas multivorans, Pseudomonas ovalis, Pseudomonas peptidolytica, Pseudomonas putida, Pseudomonas putrefaciens, Pseudomonas synxantha, Pseudomonas tabaci, Pseudomonas taetrolens, Pseudomonas umorosa, Rhizobium radiobacter, Rhizobium sp., Stenotrophomonas sp., Xanthomonas albilineans, Xanthomonas oryzae, or Yarrowia lypolytica.
[7] The production method of any of [1]-[4], wherein the L-aminotransferase consists of an amino acid sequence showing 90% or more identity to the amino acid sequence represented by SEQ ID NO:2.
[8] The production method of any of [1]-[7], wherein the 4R-IHOG is contacted with the L-aminotransferase using a transformant that expresses the L-aminotransferase.
[9] The production method of any of [1]-[8], further comprising condensing indole-3-pyruvate and pyruvate to form the 4R-IHOG.
[10] The production method of [9], the indole-3-pyruvate and the pyruvate are condensed by contacting the indole-3-pyruvate and the pyruvate with an aldolase.
[11] The production method of [9] or [10], wherein at least part of the pyruvate used in the formation of the 4R-IHOG is from pyruvate formed from the oxaloacetate due to action of the oxaloacetate decarboxylase.
[12] The production method of any of [9]-[11], further comprising oxidizing a tryptophan to form the indole-3-pyruvate.
[13] The production method of [12], wherein the tryptophan is oxidized by contacting the tryptophan with a deamination enzyme.
[14] The production method of any of [9]-[13], wherein the production of the 2S,4R-Monatin or the salt thereof is carried out in one reactor.
[15] A method for producing 2R,4R-Monatin or a salt thereof, comprising the following (I) and (II):
(I) performing the method of any of [1]-[14] to form the 2S,4R-Monatin; and
(II) isomerizing the 2S,4R-Monatin to form the 2R,4R-Monatin.
[16] The production method of [15], wherein the 2S,4R-Monatin is isomerized in the presence of an aromatic aldehyde.
[17] An L-aminotransferase that is a protein selected form the group consisting of the following (A)-(D):
(A) a protein consisting of the amino acid sequence represented by SEQ ID NO:2;
(B) a protein comprising the amino acid sequence represented by SEW ID NO:2;
(C) a protein consisting of an amino acid sequence showing 90% or more identity to the amino acid sequence represented by SEQ ID NO:2, and having an L-aminotransferase activity; and
(D) a protein consisting of an amino acid sequence comprising mutation of one or several amino acid residues, which is selected from the group consisting of deletion, substitution, addition and insertion of the amino acid residues in the amino acid sequence represented by SEQ ID NO:2, and having an L-aminotransferase activity.
[18] A polynucleotide selected from the group consisting of the following (a)-(e):
(a) a polynucleotide consisting of the nucleotide sequence represented by SEQ ID NO:1;
(b) a polynucleotide comprising the nucleotide sequence represented by SEQ ID NO:1;
(c) a polynucleotide consisting of a nucleotide sequence showing 90% or more identity to the amino acid sequence represented by SEQ ID NO:1, and encoding a protein having an L-aminotransferase activity;
(d) a polynucleotide that hybridizes under a stringent condition with a polynucleotide consisting of the nucleotide sequence complementary to the nucleotide sequence represented by SEQ ID NO:1, and encodes a protein having an L-aminotransferase activity; and
(e) a polynucleotide encoding the protein of [17].
[19] An expression vector comprising the polynucleotide of [18].
[20] A transformant introduced with the expression vector of [19].
[21] A method for producing an L-amino transfearase, comprising culturing the transformant of [20] in a medium to obtain the L-aminotransferase.
[22] A method of producing 2S,4R-Monatin or a salt thereof, comprising contacting 4R-IHOG with the L-aminotransferase of [17] in the presence of an L-amino acid to form the 2S,4R-Monatin.
[23] A method for producing 2R,4R-Monatin or a salt thereof, comprising the following (I′) and (II′):
(I′) performing the method of [22] to form the 2S,4R-Monatin; and
(II′) isomerizing the 2S,4R-Monatin to form the 2R,4R-Monatin.
[24] The production method of [23], wherein the 2S,4R-Monatin is isomerized in the presence of an aromatic aldehyde.

### Effect of the Invention

The method of the present invention can contribute to improvement of the yield of the Monatin by producing the 2S,4R-Monatin with a good yield from 4R-IHOG using the L-aminotransferase. The method of the present invention has an advantage that it is not necessary to use an expensive D-amino acid (D-Asp and the like) as a substrate when the 2S,4R-Monatin is formed from IHOG or that it is not necessary to add an enzyme such as racemase to form the D-amino acid from an L-amino acid. In the method of the present invention, when performing not only the reaction to form the 2S,4R-Monatin from 4R-IHOG (third step) but also the reaction to form IPA from L-Trp (first step) and the reaction to form 4R-IHOG from IPA (second step), whole reaction equilibrium can be defined in the third step and the reaction equilibrium in the second step can be largely shifted to a direction to form 4R-IHOG. In this case, the method of the present invention makes it possible to produce the 2S,4R-Monatin with a very good yield by avoiding a by-product of L-Trp (progress of a reverse reaction of the first step).

### BRIEF DESCRIPTION OF DRAWINGS

FIG. 1 is a view showing one example of the production method of the present invention. Trp: tryptophan; IPA: indole-3-pyruvate; IHOG: 4-(indole-3-yl-methyl)-4-hydroxy-2-oxoglutaric acid; Monatin: 4-(indole-3-yl-methyl)-4-hydroxy-glutamic acid.

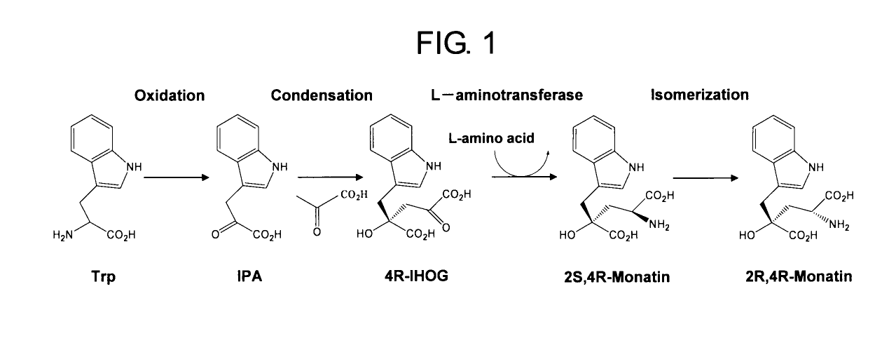

FIG. 2 is a view showing one example of the production method of the present invention. Abbreviations are the same as in FIG. 1; and

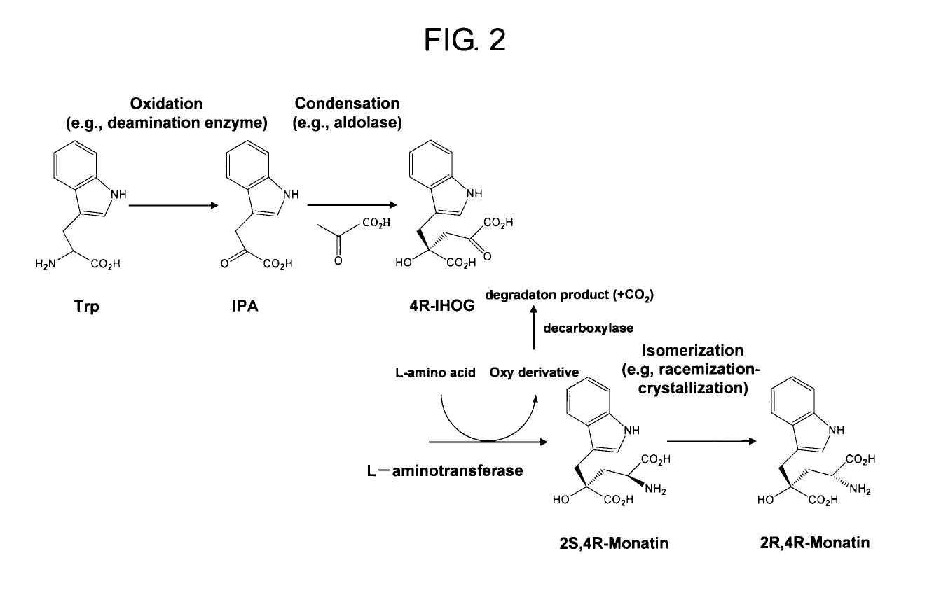

FIG. 3 is a view showing a preferable example of the production method of the present invention. L-Trp: L-tryptophan; L-Asp: L-aspartic acid; OAA: oxaloacetate; PA: pyruvate; and the other abbreviations are the same as in FIG. 1.

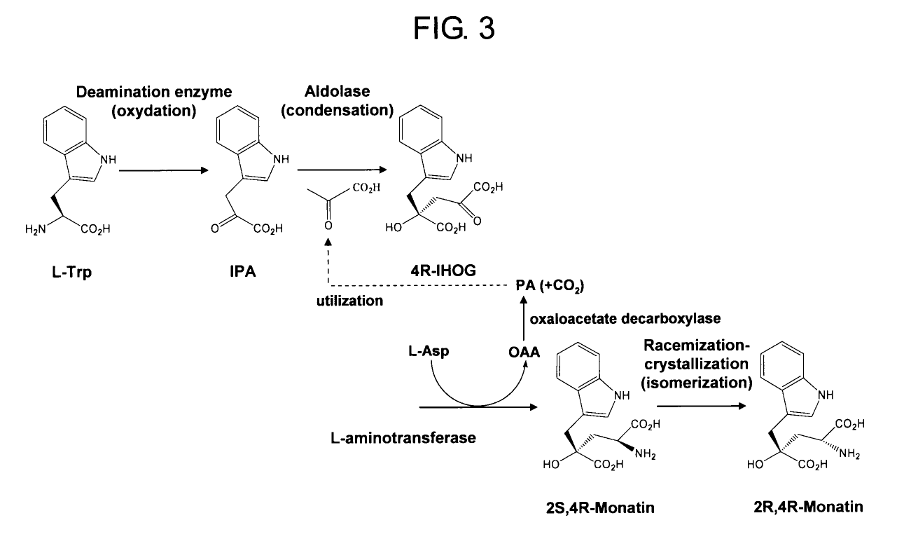

### BEST MODES FOR CARRYING OUT THE INVENTION

(1) Method for Producing 2S,4R-Monatin or a Salt Thereof

The present invention provides a method (1) for producing 2S,4R-Monatin or a salt thereof. The production method of the present invention can be classified into (1-1) a method for producing the 2S,4R-Monatin from 4R-IHOG, (1-2) a method for producing the 2S,4R-Monatin from IPA and pyruvate, and (1-3) a method for producing the 2S,4R-Monatin from tryptophan. The methods (1-1), (1-2) and (1-3) are common in contacting 4R-IHOG with an L-aminotransferase in the presence of the L-amino acid to form the 2S,4R-Monatin.

(1-1) Method for Producing 2S,4R-Monatin from 4R-IHOG

This method comprises contacting 4R-IHOG with the L-aminotransferase in the presence of the L-amino acid to form the 2S,4R-Monatin (reaction 1). By contacting 4R-IHOG with the L-aminotransferase in the presence of the L-amino acid, an amino group in the L-amino acid can be transferred to 4R-IHOG to form the 2S,4R-Monatin.

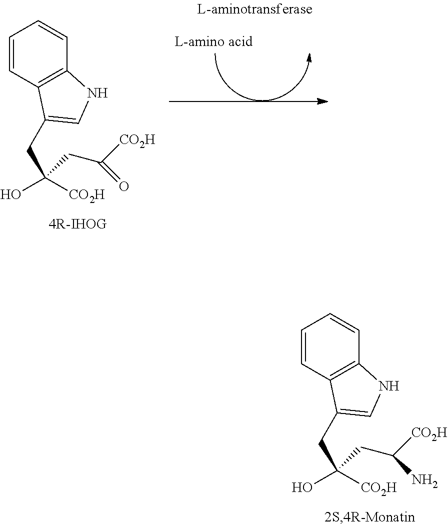

The kinds of the L-amino acid is not particularly limited as long as the amino group in the L-amino acid can be transferred to 4R-IHOG that is an objective substrate by the L-aminotransferase. Various L-amino acids such as L-α-amino acids are known as such an L-amino acid. Specifically, such an L-amino acid includes L-aspartic acid, L-alanine, L-lysine, L-arginine, L-histidine, L-glutamic acid, L-asparagine, L-glutamine, L-serine, L-threonine, L-tyrosine, L-cysteine, L-valine, L-leucine, L-isoleucine, L-proline, L-phenylalanine, L-methionine and L-tryptophan.

In one embodiment, the L-aminotransferase may be a protein derived from a microorganism such as a bacterium, actinomycete or yeast. The classification of the microorganisms can be carried out by a classification method well-known in the art, e.g., a classification method used in the database of NCBI (National Center for Biotechnology Information) (http://www.ncbi.nlm.nih.gov/Taxonomy/Browser/wwwtax.cgi?id=91347). Examples of the microorganisms from which the L-aminotransferase is derived include microorganisms belonging to genus Achromobacter, genus Alcaligenes, genus Arthrobacter, genus Bacillus, genus Candida, genus Corynebacterium, genus Lodderomyces, genus Micrococcus, genus Microbacterium, genus Nocardia, genus Pseudomonas, genus Rhizobium, genus Stenotrophomonas, genus Xanthomonas, and genus Yarrowia.

Specifically, examples of the microorganisms belonging to genus Achromobacter include Achromobacter brunificans and Achromobacter butyri. Examples of the microorganisms belonging to genus Alcaligenes include Alcaligenes faecalis and Alcaligenes metalcaligenes. Examples of the microorganisms belonging to genus Arthrobacter include Arthrobacter ureafaciens.

Examples of the microorganisms belonging to genus Bacillus include Bacillus sp. Examples of the microorganisms belonging to genus Candida include Candida norvegensis and Candida inconspicua. Examples of the microorganisms belonging to genus Corynebacterium include Corynebacterium ammoniagenes. Examples of the microorganisms belonging to genus Lodderomyces include Lodderomyces elongisporus. Examples of the microorganisms belonging to genus Micrococcus include Micrococcus luteus. Examples of the microorganisms belonging to genus Microbacterium include Microbacterium sp. Examples of the microorganisms belonging to genus Nocardia include Nocardia globerula.

Examples of the microorganisms belonging to genus Pseudomonas include Pseudomonas betainovorans, Pseudomonas chlororaphis (e.g., Pseudomonas chlororaphis subsp. chlororaphis), Pseudomonas citronocllolis, Pseudomonas fragi, Pseudomonas hydrogenovora, Pseudomonas multivorans, Pseudomonas ovalis, Pseudomonas peptidolytica, Pseudomonas putida, Pseudomonas putrefaciens, Pseudomonas synxantha, Pseudomonas tabaci, Pseudomonas taetrolens, and Pseudomonas umorosa.

Examples of the microorganisms belonging to genus Rhizobium include Rhizobium radiobacter and Rhizobium sp. Examples of the microorganisms belonging to genus Stenotrophomonas include Stenotrophomonas sp. Examples of the microorganisms belonging to genus Xanthomonas include Xanthomonas albilineans and Xanthomonas oryzae. Examples of the microorganisms belonging to genus Yarrowia include Yarrowia lypolytica.

In another embodiment, the L-aminotransferase may be a naturally occurring protein or an artificial mutant protein. Such an L-aminotransferase includes those consisting of an amino acid sequence having high homology (e.g., similarity, identity) to an amino acid sequence represented by SEQ ID NO:2, and having an L-aminotransferase activity. The term “L-aminotransferase activity” refers to an activity of transferring the amino group in the L-amino acid to 4R-IHOG that is the objective substrate for forming the 2S,4R Monatin that is an objective compound having the amino group. Specifically, the L-aminotransferase includes a protein consisting of the amino acid sequence showing 80% or more, preferably 90% or more, more preferably 95% or more and particularly preferably 98% or more or 99% or more homology (e.g., similarity, identity) to the amino acid sequence represented by SEQ ID NO:2, and having the L-aminotransferase activity.

The homology of the amino acid sequences and nucleotide sequences can be determined using algorithm BLAST by Karlin and Altschul (Pro. Natl. Acad. Sci. USA, 90, 5873 (1993)) or FASTA by Pearson (Methods Enzymol., 183, 63 (1990)). Programs referred to as BLASTP and BLASTN (see http://www.ncbi.nlm.nih.gov) have been developed based on this algorithm BLAST. Thus, the homology of the amino acid sequences and the nucleotide sequences may be calculated using these programs with default setting. A numerical value obtained when matching count is calculated as a percentage by using GENETYX Ver. 7.0.9 that is software from GENETYX Corporation and using full length polypeptide chains encoded in ORF with setting of Unit Size to Compare=2 may be used as the homology of the amino acid sequences. The lowest value among the values derived from these calculations may be employed as the homology of the amino acid sequences and the nucleotide sequences.

In further another embodiment, the L-aminotransferase may be a protein consisting of an amino acid sequence comprising mutation (e.g., deletion, substitution, addition and insertion) of one or several amino acid residues in the amino acid sequence represented by SEQ ID NO:2, and having the L-aminotransferase activity. The mutation of one or several amino acid residues may be introduced into one region or multiple different regions in the amino acid sequence. The term “one or several amino acid residues” indicate a range in which a three dimensional structure and the activity of the protein are not largely impaired. The term “one or several amino acid residues” in the case of the protein denote, for example, 1 to 100, preferably 1 to 80, more preferably 1 to 50, 1 to 30, 1 to 20, 1 to 10 or 1 to 5 amino acid residues. Such mutation may be attributed to naturally occurring mutation (mutant or variant) based on individual difference, species difference and the like of the microorganism carrying a gene encoding the L-aminotransferase.

A position of the amino acid residue to be mutated in the amino acid sequence is apparent to those skilled in the art. Specifically, a person skilled in the art can recognize the correlation between the structure and the function by 1) comparing the amino acid sequences of the multiple proteins having the same kind of activity (e.g., the amino acid sequence represented by SEQ ID NO:2, and amino acid sequences of other L-aminotransferase), 2) clarifying relatively conserved regions and relatively non-conserved regions, and then 3) predicting a region capable of playing an important role for its function and a region incapable of playing the important role for its function from the relatively conserved regions and the relatively non-conserved regions, respectively. Therefore, a person skilled in the art can specify the position of the amino acid residue to be mutated in the amino acid sequence of the L-aminotransferase.

When an amino acid residue is mutated by the substitution, the substitution of the amino acid may be conservative substitution. As used herein, the term “conservative substitution” means that a certain amino acid residue is substituted with an amino acid residue having an analogous side chain. Families of the amino acid residues having the analogous side chain are well-known in the art. Examples of such families include an amino acid having a basic side chain (e.g., lysine, arginine or histidine), an amino acid having an acidic side chain (e.g., aspartic acid or glutamic acid), an amino acid having a non-charged polar side chain (e.g., glycine, asparagine, glutamine, serine, threonine, tyrosine or cysteine), an amino acid having a non-polar side chain (e.g., alanine, valine, leucine, isoleucine, proline, phenylalanine, methionine or tryptophan), an amino acid having a β-position branched side chain (e.g., threonine, valine or isoleucine), an amino acid having an aromatic side chain (e.g., tyrosine, phenylalanine, tryptophan or histidine), an amino acid having a hydroxyl group (e.g., alcoholic or phenolic)-containing side chain (e.g., serine, threonine or tyrosine), and an amino acid having a sulfur-containing side chain (e.g., cysteine or methionine). Preferably, the conservative substitution of the amino acids may be the substitution between aspartic acid and glutamic acid, the substitution among arginine, lysine and histidine, the substitution between tryptophan and phenylalanine, the substitution between phenylalanine and valine, the substitution among leucine, isoleucine and alanine, and the substitution between glycine and alanine.

In further another embodiment, the L-aminotransferase may be a protein encoded by DNA that hybridizes under a stringent condition with a nucleotide sequence complementary to a nucleotide sequence represented by SEQ ID NO:2, and having the L-aminotransferase activity. The “stringent condition” refers to the condition where a so-called specific hybrid is formed whereas no non-specific hybrid is formed. Although it is difficult to clearly quantify this condition, one example of this condition is the condition where a pair of polynucleotides with high homology (e.g., identity), for example, a pair of polynucleotides having the homology of 80% or more, preferably 90% or more, more preferably 95% or more, and particularly preferably 90% or more are hybridized whereas a pair of polynucleotides with lower homology than that are not hybridized. Specifically, such a condition includes hybridization in 6×SSC (sodium chloride/sodium citrate) at about 45° C. followed by one or two or more washings in 0.2×SSC and 0.1% SDS at 50 to 65° C.

In one embodiment, the contact of 4R-IHOG with the L-aminotransferase can be accomplished by allowing 4R-IHOG and the L-aminotransferase extracted from an L-aminotransferase-producing microorganism (extracted enzyme) to coexist in a reaction solution. Examples of the L-aminotransferase-producing microorganism include the microorganisms that naturally produce the L-aminotransferase (e.g., the aforementioned microorganisms), and transformants that express the L-aminotransferase. Specifically, examples of the extracted enzyme include a purified enzyme, a crude enzyme, an L-aminotransferase-containing fraction prepared from the above enzyme-producing microorganism, and a disrupted product of and a lysate of the above enzyme-producing microorganism.

In another embodiment, the contact of 4R-IHOG with the L-aminotransferase can be accomplished by allowing 4R-IHOG and the L-aminotransferase-producing microorganism to coexist in the reaction solution (e.g., culture medium).

The reaction solution used in the production method (1) of the present invention is not particularly limited as long as the objective reaction progresses, and for example, water and buffer are used. Examples of the reaction solution include Tris buffer, phosphate buffer, carbonate buffer, borate buffer and acetate buffer. When the L-aminotransferase-producing microorganism is used in the production method of the present invention, the culture medium may be used as the reaction solution. Such a culture medium can be prepared using a medium described later. The reaction solution used in the production method of the present invention may further comprise pyridoxal phosphate (PLP) as a coenzyme. When the reaction solution comprises PLP, an effect to form 2R,4R-Monatin from the 2S,4R-Monatin can be expected by an isomerization reaction which can be catalyzed by PLP (e.g., see Example 11).

A pH value of the reaction solution used in the production method (1) of the present invention is not particularly limited as long as the objective reaction progresses, and is, for example, pH 5 to 10, is preferably pH 6 to 9 and is more preferably pH 7 to 8.

A reaction temperature in the production method (1) of the present invention is not particularly limited as long as the objective reaction progresses, and is, for example, 10 to 50° C., is preferably 20 to 40° C. and is more preferably 25 to 35° C.

A reaction time period in the production method (1) of the present invention is not particularly limited as long as the time period is sufficient to form the 2S,4R-Monatin, and is, for example, 2 to 100 hours, is preferably 4 to 50 hours and is more preferably 8 to 25 hours.

When a transformant that expresses the L-aminotransferase is used as the L-aminotransferase-producing microorganism, this transformant can be made by making an expression vector of the L-aminotransferase, and then introducing this expression vector into a host. For example, the transformant that expresses the L-aminotransferase can be obtained by making the expression vector incorporating DNA having the nucleotide sequence represented by SEQ ID NO:1, and introducing it into an appropriate host. For example, various prokaryotic cells including bacteria belonging to genus Escherichia such as Escherichia coli, genus Corynebacterium and Bacillus subtilis, and various eukaryotic cells including Saccharomyces cerevisiae, Pichia stipitis and Aspergillus oryzae can be used as the host for expressing the L-aminotransferase.

The hosts to be transformed are as described above. Describing Escherichia coli in detail, the host can be selected from Escherichia coli K12 strain subspecies, Escherichia coli JM109, DH5α, HB101, BL21 (DE3) strains and the like. Methods for performing the transformation and methods for selecting the transformant are described in Molecular Cloning: A Laboratory Manual, 3rd edition, Cold Spring Harbor press (2001/01/15) and the like. A method for making transformed Escherichia coli and producing a certain enzyme by the use thereof will be specifically described below as one example.

As a promoter for expressing DNA encoding the L-aminotransferase, the promoter typically used for producing a heterogeneous protein in E. coli can be used, and includes potent promoters such as T7 promoter, lac promoter, trp promoter, trc promoter, tac promoter, PR and PL promoters of lambda phage, and T5 promoter. As the vector, pUC19, pUC18, pBR322, pHSG299, pHSG298, pHSG399, pHSG398, RSF1010, pACYC177, pACYC184, pMW119, pMW118, pMW219, pMW218, pQE30 and derivatives thereof, and the like may be used. The vectors of phage DNA may also be utilized as the other vectors. Further, the expression vector containing the promoter and capable of expressing the inserted DNA sequence may be used.

A terminator that is a transcription termination sequence may be ligated to downstream of an L-aminotransferase gene. Examples of such a terminator include T7 terminator, fd phage terminator, T4 terminator, a terminator of a tetracycline resistant gene, and a terminator of an E. coli trpA gene.

So-called multiple copy types are preferable as the vector for introducing the L-aminotransferase gene into E. coli, and include plasmids having a replication origin derived from ColE1, such as pUC type plasmids, pBR322 type plasmids or derivatives thereof. Here, the “derivatives” means those in which modification is given to the plasmids by substitution, deletion, insertion, addition and/or inversion of nucleotides. The “modification” as referred to here also includes the modification by mutagenic treatments by mutagenic agents and UV irradiation, or natural mutation, or the like.

For selecting the transformant, it is preferable that the vector has a marker such as an ampicillin resistant gene. As such a plasmid, the expression vectors carrying the strong promoter are commercially available (e.g., pUC types (supplied from TAKARA BIO Inc.), pPROK types (supplied from Clontech), pKK233-2 (supplied from Clontech)).

The L-aminotransferase is expressed by transforming E. coli with the obtained expression vector and culturing this E. coli.

A medium such as M9-casamino acid medium and LB medium typically used for culturing E. coli may be used as the medium. Culture conditions and production induction conditions are appropriately selected depending on types of the marker and the promoter in the used vector, the host bacterium and the like.

The following methods and the like are available for recovering the L-aminotransferase. The L-aminotransferase can be obtained as a disrupted product or a lysate by collecting the L-aminotransferase-producing microorganism followed by disrupting (e.g., sonication, homogenization) or lysing (e.g., lysozyme treatment) the microbial cells. Also, the purified enzyme, the crude enzyme or the L-aminotransferase-containing fraction can be obtained by subjecting such a disrupted product or lysate to techniques such as extraction, precipitation, filtration and column chromatography.

In a preferred embodiment, the production method of the present invention further comprises contacting an oxy derivative of the L-amino acid (e.g., oxal derivative of L-α-amino acid: R—COCOOH) formed from the L-amino acid (e.g., L-α-amino acid) by action of the L-aminotransferase with a decarboxylase to degrade the oxy derivative of the L-amino acid (see the reaction 1′). By promoting the degradation of the oxy derivative of the L-amino acid, it is possible to shift the equilibrium of the reaction to form the 2S,4R-Monatin from 4R-IHOG so that the 2S,4R-Monatin is formed in a larger amount.

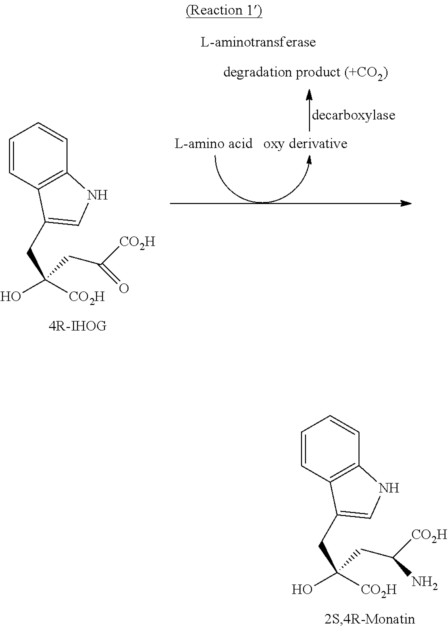

The decarboxylase used in the present invention is the enzyme that catalyzes a decarboxylation reaction of the oxy derivative of the L-amino acid. The decarboxylation reaction by the decarboxylase can be irreversible. Various enzymes are known as the decarboxylase used for the irreversible decarboxylation reaction of the oxy derivative of the L-amino acid, and examples thereof include an oxaloacetate decarboxylase derived from Pseudomonas stutzeri (Arch Biochem Biophys., 365, 17-24, 1999) and a pyruvate decarboxylase derived from Zymomonas mobilis (Applied Microbiology and Biotechnology, 17, 152-157, 1983).

In a particularly preferred embodiment, the production method of the present invention comprises contacting oxaloacetate (OAA) formed from L-aspartic acid (L-Asp) by action of the L-aminotransferase with the oxaloacetate decarboxylase to form the pyruvate (PA) (see the reaction 1″). By promoting the irreversible formation of the pyruvate from the oxaloacetate, it is possible to shift the equilibrium of the reaction to form the 2S,4R-Monatin from 4R-IHOG so that the 2S,4R-Monatin is formed in a larger amount.

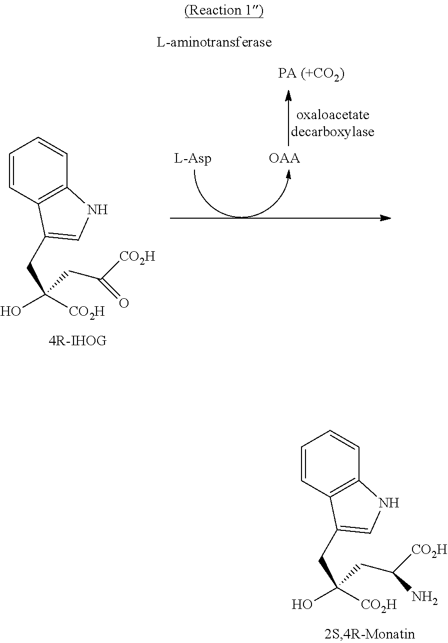

The oxaloacetate decarboxylase used in the present invention is the enzyme that catalyzes the decarboxylation reaction of the oxaloacetate to form the pyruvate. The decarboxylation reaction by the oxaloacetate decarboxylase can be irreversible. Various enzymes are known as the oxaloacetate decarboxylase used for the irreversible decarboxylation reaction of the oxaloacetate. Examples of such an oxaloacetate decarboxylase include the oxaloacetate decarboxylase derived from Pseudomonas stutzeri (Arch Biochem Biophys., 365, 17-24, 1999), the oxaloacetate decarboxylase derived from Klebsiella aerogenes (FEBS Lett., 141, 59-62, 1982), and the oxaloacetate decarboxylase derived from Sulfolobus solfataricus (Biochim Biophys Acta., 957, 301-311, 1988).

When the decarboxylase is used in the production of the 2S,4R-Monatin from 4R-IHOG, the contact of the oxy derivative of the L-amino acid with the decarboxylase can be accomplished by allowing the oxy derivative of the L-amino acid and the decarboxylase extracted from a decarboxylase-producing microorganism (extracted enzyme) or the decarboxylase-producing microorganism to coexist in the reaction solution (e.g., culture medium). Examples of the decarboxylase-producing microorganism include microorganisms that naturally produce the decarboxylase and transformants that express the decarboxylase. Examples of the extracted enzyme include a purified enzyme, a crude enzyme, a decarboxylase-containing fraction prepared from the above decarboxylase-producing microorganism, and a disrupted product of and a lysate of the above decarboxylase-producing microorganism.

When both the L-aminotransferase and the decarboxylase are used in the production of the 2S,4R-Monatin from 4R-IHOG, the L-aminotransferase and the decarboxylase may be provided in the reaction solution in the following manner:

L-aminotransferase (extracted enzyme) and decarboxylase (extracted enzyme);

L-aminotransferase-producing microorganism and decarboxylase (extracted enzyme);

L-aminotransferase (extracted enzyme) and decarboxylase-producing microorganism;
L-aminotransferase-producing microorganism and decarboxylase-producing microorganism; and

L-aminotransferase- and decarboxylase-producing microorganism.

Preferably, the L-aminotransferase- and decarboxylase-producing microorganism may be a transformant. Such a transformant can be made by i) introducing an expression vector of the L-aminotransferase into the decarboxylase-producing microorganism, ii) introducing an expression vector of the decarboxylase into the L-aminotransferase-producing microorganism, (iii) introducing a first expression vector of the L-aminotransferase and a second expression vector of the decarboxylase into a host microorganism, and (iv) introducing an expression vector of the L-aminotransferase and the decarboxylase into the host microorganism. Examples of the expression vector of the L-aminotransferase and the decarboxylase include i′) an expression vector containing a first expression unit composed of a first polynucleotide encoding the L-aminotransferase and a first promoter operatively linked to the first polynucleotide, and a second expression unit composed of a second polynucleotide encoding the decarboxylase and a second promoter operatively linked to the second polynucleotide; and ii') an expression vector containing a first polynucleotide encoding the L-aminotransferase, a second polynucleotide encoding the decarboxylase and a promoter operatively linked to the first polynucleotide and the second polynucleotide (vector capable of expressing polycistronic mRNA). The first polynucleotide encoding the L-aminotransferase may be located upstream or downstream the second polynucleotide encoding the decarboxylase.

(1-2) Method for Producing 2S,4R-Monatin from IPA and Pyruvate

The production method of the present invention may further comprise condensing IPA and the pyruvate to form 4R-IHOG in order to prepare 4R-IHOG. The condensation of IPA and the pyruvate can be carried out by the organic chemistry process, or an enzymatic method using an aldolase. The method for forming 4R-IHOG by condensing IPA and the pyruvate by the organic chemistry process is disclosed in, for example, International Publication WO2003/059865 and US Patent Application Publication No. 2008/0207920. The method for forming 4R-IHOG by condensing IPA and the pyruvate by the enzymatic method using the aldolase is disclosed in, for example, International Publication WO2003/056026, JP 2006-204285-A, US Patent Application Publication No. 2005/0244939 and International Publication WO2007/103989. Therefore, in the present invention, these methods can be used in order to prepare 4R-IHOG from IPA and the pyruvate.

IPA used for the preparation of 4R-IHOG is an unstable compound. Therefore, the condensation of IPA and the pyruvate may be carried out in the presence of a stabilizing factor for IPA. Examples of the stabilizing factor for IPA include superoxide dismutase (e.g., see International Publication WO2009/028338) and mercaptoethanol (e.g., see International Publication WO2009/028338). For example, the transformant expressing the superoxide dismutase is disclosed in International Publication WO2009/028338. Thus, such a transformant may be used in the method of the present invention.

The reaction to form 4R-IHOG from IPA and the pyruvate and the reaction to form the 2S,4R-Monatin from 4R-IHOG may be progressed separately or in parallel. These reactions may be carried out in one reactor. When these reactions are carried out in one reactor, these reactions can be carried out by adding the substrates and the enzymes sequentially or simultaneously. Specifically, when the reaction to form 4R-IHOG from IPA and the pyruvate by the enzymatic method using the aldolase and the reaction to form the 2S,4R-Monatin from 4R-IHOG by the L-aminotransferase are carried out, (1) IPA, the pyruvate and the aldolase, and (2) the L-amino acid and the L-aminotransferase may be added in one reactor sequentially or simultaneously.

In a preferred embodiment, the production method of the present invention is combined with the above reaction 1″ as follows. In this case, the pyruvate irreversibly formed from the oxaloacetate is utilized for the preparation of 4R-IHOG. In other words, at least a part of the pyruvate used for the formation of 4R-IHOG can be from the pyruvate formed from the oxaloacetate by action of the oxaloacetate decarboxylase. In this case, it should be noted that an initial amount of the pyruvate in the reaction system is not necessarily important if an amount of the L-amino acid present in the reaction system is sufficient because the pyruvate is formed from the oxaloacetate in conjunction with the formation of the 2S,4R-Monatin. Therefore, the larger amount of the L-amino acid may be added to the reaction system compared with the pyruvate.

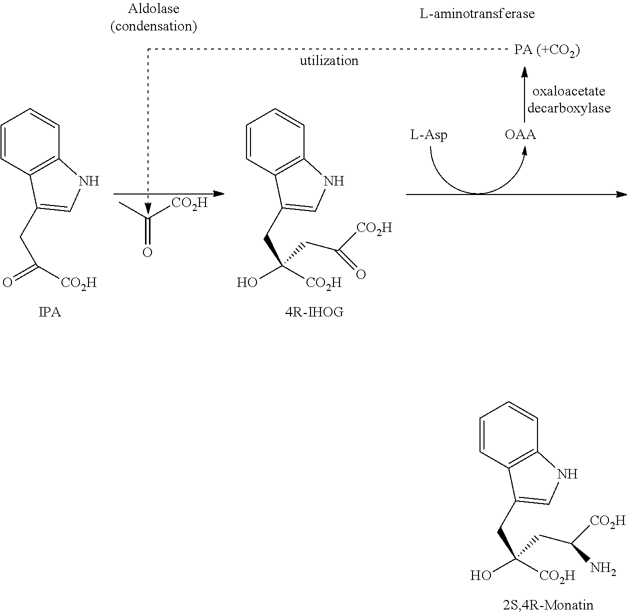

When the aldolase is used in the production of 4R-IHOG from IPA and the pyruvate, the contact of IPA and the pyruvate with the aldolase can be accomplished by allowing IPA, the pyruvate and the aldolase extracted from an aldolase-producing microorganism (extracted enzyme) or the aldolase-producing microorganism to coexist in the reaction solution (e.g., culture medium). Examples of the aldolase-producing microorganism include microorganisms that naturally produce the aldolase and transformants that express the aldolase. Examples of the extracted enzyme include a purified enzyme, a crude enzyme, an aldolase-containing fraction prepared from the above aldolase-producing microorganism, a disrupted product of and a lysate of the above aldolase-producing microorganism. The aldolase-producing microorganism may further express other enzyme(s) (e.g., superoxide dismutase, L-aminotransferase, decarboxylase). Alternatively, a microorganism that produces the other enzyme in addition to the aldolase-producing microorganism may be allowed to coexist in the reaction solution. Those described in the production method (1-1) of the present invention can be used as the reaction solution.

Various conditions such as the temperature, the pH value and the time period in the reaction can be appropriately established as long as the objective reaction can progress. For example, the conditions of the enzymatic method using the aldolase may be the same as those described in the production method (1-1) of the present invention.

(1-3) Method for Producing 2S,4R-Monatin or a Salt Thereof from Tryptophan or a Salt Thereof

The production method of the present invention may further comprise oxidizing a tryptophan (Trp) in order to prepare IPA. Trp includes L-Trp, D-Trp and a mixture of L-Trp and D-Trp. The oxidation of Trp can be performed by the organic chemistry technique and the enzymatic method using a deamination enzyme.

Various methods are known as the method for oxidizing Trp to form IPA by the organic chemistry technique. Examples of such a method include the method in which the tryptophan is used as a starting material and reacted with pyridine aldehyde in the presence of a base for dehydration of a proton acceptor (e.g., see JP Sho-62-501912 and International Publication WO1987/000169), and the method of subjecting to acid hydrolysis after a condensation reaction using indole and ethyl-3-bromopyruvate ester oxime as raw materials (e.g., European Patent Application Publication No. 421946).

As used herein, the term “deamination enzyme” refers to the enzyme capable of forming IPA from Trp. The formation of IPA from Trp is essentially conversion of the amino group (—NH₂) in Trp to an oxy group (═O). Therefore, the enzymes that catalyze this reaction are sometimes termed as other names such as an amino acid deaminase, an aminotransferase and an amino acid oxidase. Therefore, the term “deamination enzyme” means any enzyme that can form IPA from Trp, and the enzymes having the other name (e.g., amino acid deaminase, aminotransferase, amino acid oxidase) which catalyze the reaction to form IPA from Trp are also included in the “deamination enzyme.”

Examples of the method for forming IPA from Trp using the amino acid deaminase or an amino acid deaminase-producing microorganism include the method disclosed in International Publication WO2009/028338. A general formula of the reaction catalyzed by the amino acid deaminase includes the following formula: Amino acid+H₂O→2-oxo acid+NH₃.

Examples of the method for forming IPA from Trp using the aminotransferase or an aminotransferase-producing microorganism include the methods disclosed in East Germany Patent DD 297190, JP Sho-59-95894-A, International Publication WO2003/091396 and US Patent Application Publication No. 2005/028226.

Examples of the method for forming IPA from Trp using the L-amino acid oxidase or an L-amino acid oxidase-producing microorganism include the methods disclosed in U.S. Pat. No. 5,002,963, John A. Duerre et al. (Journal of Bacteriology 1975, vol. 121, No. 2, p656-663), JP Sho-57-146573, International Publication WO2003/056026 and International Publication WO2009/028338. The general formula of the reaction catalyzed by the amino acid oxidase includes the following formula: Amino acid+O₂+H₂O→+2-Oxo acid+H₂O₂+NH₃. For the purpose of suppressing the degradation of the compound by hydrogen peroxide as the by-product produced at that time, a hydrogen peroxide-degrading enzyme such as a catalase may be added in the reaction solution.

The reaction to form IPA from Trp, the reaction to form 4R-IHOG from IPA and the pyruvate and the reaction to form 2S,4R-Monatin from 4R-IHOG may be progressed separately or in parallel. These reactions may be carried out in one reactor. When these reactions are carried out in one reactor, these reactions can be carried out by adding the substrates and the enzymes sequentially or simultaneously. Specifically, when the reaction to oxidize Trp by the enzymatic method using the deamination enzyme to form IPA, the reaction to form 4R-IHOG from IPA and the pyruvate by the enzymatic method using the aldolase, and the reaction to form 2S,4R-Monatin from 4R-IHOG by the L-aminotransferase are carried out, (1) Trp and the deamination enzyme, (2) the pyruvate and the aldolase, and (3) the L-amino acid and the L-aminotransferase may be added in one reactor sequentially or simultaneously.

When the deamination enzyme is used in the production of IPA from Trp, the contact of Trp with the deamination enzyme can be accomplished by allowing Trp and the deamination enzyme extracted from a deamination enzyme-producing microorganism (extracted enzyme) or the deamination enzyme-producing microorganism to coexist in the reaction solution. Examples of the deamination enzyme-producing microorganism include microorganisms that naturally produce the deamination enzyme and transformants that express the deamination enzyme. Examples of the extracted enzyme include a purified enzyme, a crude enzyme, a deamination enzyme-containing fraction prepared from the above deamination enzyme-producing microorganism, a disrupted product of and a lysate of the above deamination enzyme-producing microorganism. The deamination enzyme-producing microorganism may further express the other enzyme(s) (e.g., aldolase, superoxide dismutase, L-aminotransferase, decarboxylase). Alternatively, the other enzyme-producing microorganism in addition to the deamination enzyme-producing microorganism may be allowed to coexist in the reaction solution. Those described in the production method (1-1) of the present invention can be used as the reaction solution.

Various conditions such as the temperature, the pH value and the time period in the reaction can be appropriately established as long as the objective reaction can progress. For example, the conditions of the enzymatic method using the deamination enzyme may be the same as those described in the production method (1-1) of the present invention.

The purified 2S,4R-Monatin can be obtained by taking advantage of known purification methods such as column treatment, crystallization treatment and extraction treatment for a 2S,4R-Monatin-containing reaction solution obtained by any of the production methods (1-1), (1-2) and (1-3) of the present invention. The purified 2S,4R-Monatin can be provided to a method (2) for producing 2R,4R-Monatin or a salt thereof. The 2S,4R-Monatin-containing reaction solution obtained by any of the production methods (1-1), (1-2) and (1-3) of the present invention can also be directly provided to the method (2) for producing the 2R,4R-Monatin or the salt thereof.

(2) Method for Producing 2R,4R-Monatin or a Salt Thereof

The present invention provides a method (2) for producing 2R,4R-Monatin or the salt thereof. The production method of the present invention comprises performing the production method (1) of the present invention to form the 2S,4R-Monatin or a salt thereof, and isomerizing the 2S,4R-Monatin or the salt thereof to form 2R,4R-Monatin or a salt thereof.

The isomerization of the 2S,4R-monatin to the 2R,4R-Monatin can be performed by any method that enables the isomerization (e.g., see International Publication WO2005/082850 and International Publication WO03/059865). However, in terms of enhancing a yield of the 2R,4R-Monatin, the isomerization of the 2S,4R-Monatin is preferably performed by epimerization-crystallization (e.g., see International Publication WO2005/082580). The epimerization-crystallization is a method in which the isomerization reaction and the crystallization are performed simultaneously. In this case, the isomerization reaction at position 2 to convert the 2S,4R-Monatin into the 2R,4R-Monatin and the crystallization of the converted 2R,4R-Monatin are performed simultaneously by the epimerization-crystallization.

In the epimerization-crystallization, the isomerization reaction may be performed in the presence of an aldehyde. The aldehyde includes an aliphatic aldehyde and an aromatic aldehyde, and the aromatic aldehyde is preferred. A purified 2S,4R-Monatin or a 2S,4R-Monatin-containing reaction solution may be used as the 2S,4R-Monatin used for the isomerization reaction.

For the aliphatic aldehyde, for example, a saturated or unsaturated aldehyde having 1 to 7 carbon atoms, such as formaldehyde, acetaldehyde, propionaldehyde, n-butyl aldehyde, 1-butyl aldehyde, n-valeraldehyde, capronaldehyde, n-heptylaldehyde, acrolein or methacrolein can be used.

For the aromatic aldehyde, the aromatic aldehyde such as benzaldehyde, salicylaldehyde, m-hydroxybenzaldehyde, p-hydroxybenzaldehyde, o-nitrobenzaldehyde, p-nitrobenzaldehyde, 5-nitrosalicylaldehyde, 3,5-dichlorosalicylaldehyde, anisaldehyde, o-vanillin, vanillin, furfural, pyridoxal or 5-phosphate pyridoxal can be used. Particularly, pyridoxal, 5-nitrosalicylaldehyde, or 3,5-dichlorosalicylaldehyde is preferred as the aromatic aldehyde.

The aldehyde can be used in the range of 0.01 to 1 mol equivalent and more preferably 0.05 to 0.5 mol equivalent to the Monatin present in the system.

The epimerization-crystallization is performed in the presence of the aldehyde, and a mixed solvent of water and an organic solvent is used as a solvent. The organic solvent miscible with the water is used as the organic solvent, and particularly, alcohol such as methanol, ethanol, propanol or isopropanol is preferred. Two or more different kinds of organic solvents may be used in mixture. A volume ratio of the organic solvent to the water is set in the range of preferably 1:0.01 to 1:1 and more preferably 1:0.1 to 1:0.5 (organic solvent:water).

The temperature in the epimerization-crystallization is set in the range of preferably 0 to 100° C. and more preferably 40 to 80° C. The time period for performing the epimerization-crystallization is set in the range of preferably 10 hours to one week and more preferably 15 hours to 96 hours.

The pH value is set in the range of 4 to 13, preferably 4.5 to 10 and more preferably 5 to 9. The pH value can be adjusted using an acid or an alkali. The acid to be used is not particularly limited, and an organic acid such as acetic acid, or an inorganic acid such as hydrochloric acid or sulfuric acid can be used. The alkali is not also particularly limited, and an alkali metal hydroxide such as sodium hydroxide or potassium hydroxide, or an organic base such as ammonia or amine can be used.

Each compound obtained by the above method can be isolated and purified by known separation and purification procedures such as concentration, reduced pressure concentration, solvent extraction, crystallization, recrystallization, solvent transfer and chromatography. The salts of the compound used in the method of the present invention and the compound (objective compound) produced by the method of the present invention can be produced, for example, by adding the inorganic acid or the organic acid to the objective compound according to the method publicly known per se. The objective compound and the salt thereof may be hydrate, and both hydrate and non-hydrate are included in the scope of the present invention. The compounds (e.g., Trp, IPA, 4R-IHOG, 2S,4R-Monatin) used for the production methods of the present invention may be the forms of various salts such as sodium salts, potassium salts and ammonium salts. The compounds (e.g., IPA, 4R-IHOG, 2S,4R-Monatin, 2R,4R-Monatin) obtained by the production method of the present invention may also be the forms of various salts.

The present invention will be described in detail by the following Examples, but the present invention is not limited by these Examples.

### EXAMPLES

(Analytical Condition of HPLC)

In Examples 1 to 7, if HPLC analysis was performed, the HPLC analysis was performed under the condition shown in the Example.

In Examples 8 to 15, the HPLC analysis was performed under the condition shown below.

Detector: Ultraviolet absorption spectrometer (measured wavelength: 210 nm)

Column temperature: 40° C.

Column: CAPCELLPAK C18 Type MGII, inner diameter: 3 mm, length: 25 cm, and particle diameter: 5 μm, Shiseido Co., Ltd.

Mobile phase: Solution A (aqueous solution of 20 mM potassium dihydrogen phosphate:acetonitrile=95:5) and solution B (aqueous solution of 20 mM potassium dihydrogen phosphate:acetonitrile=60:40)

Gradient program: See the following Table 1

TABLE 1
Gradient program

| Time (min) | Mobile phase A (%) | Mobile phase B (%) |
|---|---|---|
| 0.0 | 100 | 0 |
| 15.0 | 100 | 0 |
| 40.0 | 0 | 100 |
| 45.0 | 0 | 100 |
| 45.1 | 100 | 0 |

Flow: 0.45 mL/minute

Injection amount: 20 μL

Analysis time period: 60 minutes

### Example 1

### Formation of 2S,4R-Monatin from 4R-IHOG Using Extraction Solution from Bacillus sp. AJ1616 Microbial Cells

Bacillus sp. AJ1616 was streaked on CM2G agar medium (10 g/L of yeast extract, 10 g/L of polypeptone, 5 g/L of glucose, 5 g/L of sodium chloride, 15 g/L of agar, pH 7.0), and cultured at 30° C. for 2 days.

One loopful of the resulting microbial cells was inoculated to 3 mL of an enzyme production medium (10 g/L of yeast extract, 10 g/L of polypeptone, 1 g/L of glucose, 3 g/L of dipotassium hydrogen phosphate, 1 g/L of potassium dihydrogen phosphate, 0.1 g/L of magnesium sulfate heptahydrate, 5 g/L of ammonium sulfate) in a test tube, which was then cultured with shaking at 30° C. for 16 hours. The microbial cells were collected from 2 mL of the cultured medium by centrifugation, washed with and suspended in 20 mM Tris-HCl (pH 7.6) to prepare 1 mL of a microbial cell suspension.

1 g of glass beads (0.1 mm) was added to 1 mL of this microbial cell suspension, and the microbial cells were disrupted using a multi beads shocker (Yasui Kikai Co., Ltd.). The resulting disrupted cell solution was centrifuged to use a supernatant as a microbial cell extract.

A 2S,4R-Monatin synthesis reaction solution (0.1 mL) (9.5 mM 4R-IHOG, 0.5 mM 4S-IHOG, 100 mM L-Asp, 50 μM PLP, 100 mM Tris-HCl, pH 8.0) was prepared so that 0.05 mL of the Bacillus sp. AJ1616 microbial cell extract was contained. The reaction solution was reacted at 30° C. for 20 hours. After termination of the reaction, the formed 2S,4R-Monatin was quantified, and its concentration was 0.21 mM.

The 2S,4R-Monatin was quantified using HPLC (Waters). The analytical condition is as follows.

Mobile phase: 20 mM KH₂PO₄/asetonitrile=100/5

Flow rate: 0.15 mL/minute

Column temperature: 40° C.

Detection: UV 210 nm

Column: ACQUITY HPLC BEH C18, 2.1×50 mm, 1.7 μm (Waters).

### Example 2

### Purification of Aminotransferase Derived from Bacillus sp. AJ1616

An aminotransferase for forming the 2S,4R-Monatin was purified from a soluble fraction of Bacillus sp. AJ1616 as follows. The reaction for synthesizing 2S,4R-Monatin and the quantification of 2S,4R-Monatin were performed in the same manner as in Example 1.

(1) Preparation of Soluble Fraction

Bacillus sp. AJ1616 was streaked on CM2G agar medium (10 g/L of yeast extract, 10 g/L of polypeptone, 5 g/L of glucose, 5 g/L of sodium chloride, 15 g/L of agar, pH 7.0), and cultured at 30° C. for 2 days.

One loopful of the resulting microbial cells was inoculated to 160 mL of TB (Terrific Broth) medium in a 500 mL Sakaguchi flask, which was then cultured with shaking at 30° C. for 16 hours. The microbial cells were collected from about 2000 mL of the cultured medium by centrifugation, washed with and suspended in 20 mM Tris-HCl (pH 7.6), 100 mM NaCl, and then disrupted by sonication at 4° C. for 30 minutes. Microbial cell debris was removed from the disrupted solution by centrifugation, and the resulting supernatant was used as a soluble fraction.

(2) Anion Exchange Chromatography

The above soluble fraction was applied onto an anion exchange chromatography column HiLoad 26/10 Q Sepharose HP (supplied from GE Health Care Bioscience, CV=53 mL) equilibrated with 20 mM Tris-HCl (pH 7.6), 100 mM NaCl, and adsorbed to the carrier. Proteins that had not been adsorbed to the carrier (unadsorbed proteins) were washed out with 20 mM Tris-HCl (pH 7.6), 100 mM NaCl, and subsequently the adsorbed proteins were eluted by linearly changing the concentration of NaCl from 100 mM to 500 mM at a flow rate of 8 mL/minute. A 2S,4R-Monatin forming activity was measured in each fraction, and detected in the fractions corresponding to about 200 mM NaCl.

(3) Hydrophobic Chromatography

The fractions in which the 2S,4R-Monatin forming activity had been detected were combined, and ammonium sulfate and Tris-HCl (pH 7.6) were added thereto at final concentrations of 1.4 M and 20 mM, respectively. This solution was applied to a hydrophobic chromatography column HiLoad 16/10 Phenyl Sepharose HP (supplied from GE Health Care Bioscience, CV=20 mL) equilibrated with 1.4 M ammonium sulfate, 20 mM Tris-HCl (pH 7.6), and adsorbed to the carrier. Unadsorbed proteins that had not been adsorbed to the carrier were washed out with 1.4 M ammonium sulfate, 20 mM Tris-HCl (pH 7.6), and subsequently, a 2S,4R-Monatin forming enzyme was eluted by linearly changing the concentration of ammonium sulfate from 1.4 M to 0 M at a flow rate of 3 mL/minute. The 2S,4R-Monatin forming activity was measured in each fraction, and detected in the fractions corresponding to about 1.0 M ammonium sulfate.

(4) Gel Filtration Chromatography

The fractions in which the 2S,4R-Monatin forming activity had been detected were combined and concentrated using Amicon Ultra-15 30K (Millipore). The resulting concentrated solution was diluted with 20 mM Tris-HCl (pH 7.6), 150 mM NaCl. This solution was applied to a gel filtration column HiLoad 16/60 Superdex 200 pg (supplied from GE Health Care Bioscience, CV=120 mL) equilibrated with 20 mM Tris-HCl (pH 7.6), 150 mM NaCl, and eluted at a flow rate of 1 mL/minute. This manipulation confirmed the 2S,4R-Monatin forming activity in a location estimated as a molecular weight of about 120 kDa.

(5) Anion Exchange Chromatography

The fractions in which the 2S,4R-Monatin forming activity had been detected were combined and applied to an anion exchange chromatography column Mono Q 5/5 (supplied from Pharmacia (GE Health Care Bioscience), CV=1 mL) equilibrated with 20 mM Tris-HCl, 100 mM NaCl (pH 7.6), and adsorbed to the carrier. Proteins that had not been adsorbed to the carrier (unadsorbed proteins) were washed out with 20 mM Tris-HCl (pH 7.6), 100 mM NaCl, and subsequently the adsorbed proteins were eluted by linearly changing the concentration of NaCl from 100 mM to 500 mM at a flow rate of 0.5 mL/minute. The 2S,4R-Monatin forming activity was measured in each fraction, and detected in the fractions corresponding to about 200 mM NaCl.

(6) SDS-PAGE

The obtained fractions were subjected to SDS-PAGE, and a band around 45 kDa was observed in the active fraction. This band was subjected to analysis of an N-terminal amino acid sequence as a candidate for the aminotransferase for forming the 2S,4R-Monatin. The band was also subjected to the analysis of an internal amino acid sequence.

### Example 3

### Determination of N-Terminal and Internal Amino Acid Sequences of Aminotransferase Derived from Bacillus sp AJ1616

The purified enzyme solution obtained in Example 2 was subjected to the analysis of the N-terminal amino acid sequence, and the sequence SGFTALSEAELNDLY (SEQ ID NO:4) was obtained as the N-terminal amino acid sequence. The sample in SDS-PAGE gel was treated with trypsin (pH 8.0, 35° C., 20 hours), and subsequently subjected to reverse phase HPLC to separate peptide fragments. The amino acid sequences in the fractionated fractions were analyzed, and the sequence QLDLSMGMLDVV (SEQ ID NO:5) was obtained as the internal amino acid sequence. Both the N-terminal amino acid sequence and the internal amino acid sequence exhibited high homology to the aminotransferase derived from Bacillus pumilus SAFR-032 (YP_{—}001487343).

### Example 4

### Cloning of Aminotransferase Gene Derived from Bacillus sp. AJ1616

Bacillus sp. AJ1616 was cultured in the same manner as in Example 1. The microbial cells were collected from the cultured medium by centrifugation, and genomic DNA was extracted.

A DNA fragment including an aminotransferase gene was amplified by PCR using the obtained genomic DNA as a template. For primers, the primer Bp-u300-f (5′-ctcaggaagcaggcgcaaaaagattaattt-3′ (SEQ ID NO:6) and the primer Bp-d200-r (5′-ggatgctgtctttgtcatcccaaagtggat-3′ (SEQ ID NO:7) were used, which were designed from DNA sequences of upstream 300 bp and downstream 200 bp in the aminotransferase gene with reference to the genomic DNA sequence of Bacillus pumilus SAFR-032 (CP000813). PCR was performed using KOD-plus-ver. 2 (Toyobo) under the following condition.

| | |
|---|---|
| 1 cycle | 94° C., 2 min |
| 25 cycles | 98° C., 10 sec |
| | 55° C., 10 sec |
| | 68° C., 60 sec |
| 1 cycle | 68° C., 60 sec |
| | 4° C. |

A nucleotide sequence of about 1800 bp of the amplified DNA fragment was determined, and the nucleotide sequence was shown to include 1308 bp of ORF that had the high homology to the aminotransferase gene derived from Bacillus pumilus SAFR-032 (NC_{—}009848). The homology was 89% in the DNA sequences and 93% in the amino acid sequences.

The N-terminal amino acid sequence and the internal amino acid sequence obtained in Example 3 were found in this sequence. Thus, it was thought that the aminotransferase gene having the 2S,4R-Monatin forming activity could have been acquired.

### Example 5

### Expression of Aminotransferase Derived from Bacillus sp. AJ1616 in E. coli

(1) Construction of Plasmid Expressing Aminotransferase Derived from Bacillus sp. AJ1616

A DNA fragment including the aminotransferase gene derived from Bacillus sp. AJ1616 was amplified by PCR using the genomic DNA of Bacillus sp. AJ1616 as the template. The primer 1616AT-Nde-f (5′-ggaattccatATGAGCGGTTTTACAGCGTT-3′: SEQ ID NO:8) and the primer 1616-xho-r (5′-gtcaaggagtttttctcgagTACCGTTGGTGCTGATTGAC-3′: SEQ ID NO:9) were used as the primers. A NdeI sequence in the aminotransferase gene was converted using the primer 1616-delNde-f (5′-GGATTGAAGGAACAcATGAAAAAGCATGC-3′: SEQ ID NO:10) and the primer 1616-delNde-r (5′-GCATGCTTTTTCATgTGTTCCTTCAATCC-3′: SEQ ID NO:11). PCR was performed using KOD-plus-ver. 2 (Toyobo) under the following condition.

| | |
|---|---|
| 1 cycle | 94° C., 2 min |
| 25 cycles | 98° C., 10 sec |
| | 55° C., 10 sec |
| | 68° C., 60 sec |
| 1 cycle | 68° C., 60 sec |
| | 4° C. |

The resulting DNA fragment of about 1300 bp was treated with restriction enzymes NdeI and XhoI, and then ligated to pET-22b (Novagen) likewise treated with NdeI and XhoI. E. coli JM109 was transformed with this solution containing the ligated product, the objective plasmid was extracted from ampicillin resistant colonies, and this plasmid was designated as pET-22-1616AT-His. This plasmid expresses the aminotransferase derived from Bacillus sp. AJ1616 having His-tag at C-terminus (1616AT-His).

(2) Purification of 1616AT-His from E. coli Expression Strain

The constructed expression plasmid pET-22-1616AT-His was introduced into E. coli BL21 (DE3). One loopful of the resulting transformant was inoculated to 160 mL of Overnight Express Instant TB Medium (Novagen) containing 100 mg/L of ampicillin in a 500 mL Sakaguchi flask, and cultured with shaking at 37° C. for 16 hours. After the termination of the cultivation, microbial cells were collected from about 1000 mL of the resulting cultured medium by centrifugation, washed with and suspended in 20 mM Tris-HCl (pH 7.6), 100 mM NaCl and 20 mM imidazole, and disrupted by sonication at 4° C. for 30 minutes. Microbial cell debris was removed from the disrupted solution by centrifugation, and the resulting supernatant was used as a soluble fraction.

The obtained soluble fraction was applied to a His-tag protein purification column His Prep FF 16/10 (supplied from Pharmacia (GE Health Care Bioscience), CV=20 mL) equilibrated with 20 mM Tris-HCl (pH 7.6), 100 mM NaCl and 20 mM imidazole, and adsorbed to the carrier. Proteins that had not been adsorbed to the carrier (unadsorbed proteins) were washed out with 20 mM Tris-HCl (pH 7.6), 100 mM NaCl and 20 mM imidazole, and subsequently the adsorbed proteins were eluted by linearly changing the concentration of imidazole from 20 mM to 250 mM at a flow rate of 3 mL/minute.

The obtained fractions were combined and concentrated using Amicon Ultra-15 30K (Millipore). The concentrated solution was diluted with 20 mM Tris-HCl (pH 7.6), 100 mM NaCl, and applied to the anion exchange chromatography column HiLoad 16/10 Q Sepharose HP (supplied from GE health Care Bioscience, CV=20 mL) equilibrated with 20 mM Tris-HCl (pH 7.6), 100 mM NaCl, and adsorbed to the carrier. The proteins that had not been adsorbed to the carrier (unadsorbed proteins) were washed out with 20 mM Tris-HCl (pH 7.6), 100 mM NaCl, and subsequently the adsorbed proteins were eluted by linearly changing the concentration of NaCl from 100 mM to 500 mM at a flow rate of 3 mL/minute.

The 2S,4R-Monatin forming activity was measured in each eluted fraction, and the fractions in which the 2S,4R-Monatin forming activity had been confirmed were combined and concentrated using Amicon Ultra-15 30K (Millipore). The concentrated solution was diluted with 20 mM Tris-HCl (pH 7.6) to use as a 1616AT-His solution.

### Example 6

### Synthesis Reaction of 2S,4R-Monatin Using 1616AT-His

The 2S,4R-Monatin was quantified by HPLC analysis. The analytical condition was as follows.

Mobile phase: 20 mM KH₂PO₄/acetonitrile=100/5

Flow rate: 1.0 mL/minute

Column temperature: 40° C.

Detection: UV 280 nm

Column: CAPCELL PAK MGII, 4.6×150 mm, 3 μm, (Shiseido Co., Ltd.)

(1) Synthesis of 2S,4R-Monatin from 4R-IHOG

The 1616AT-His solution prepared so as to contain 0.5 mg of 1616AT-His (Example 5) was added to 0.1 mL of the reaction solution (9.5 mM 4R-IHOG, 0.5 mM 4S-IHOG, 80 mM L-Asp, 50 μM PLP, 100 mM Tris-HCl, pH 8.0), and then reacted at 25° C. for 12 hours. After the termination of the reaction, the formed 2S,4R-Monatin was quantified, and its concentration was 8.6 mM.

(2) Synthesis of 2S,4R-Monatin from Indole Pyruvate (IPA) and Pyruvate (PA)

A reaction mixture was prepared so as to contain 0.5 mg of 1616AT-His (the 1616AT-His solution in Example 5 was used), 0.01 mg of SpAld (JP 2006-204285-A) and 1 U of oxaloacetate decarboxylase (Sigma, O4878) in 0.1 mL of a reaction solution (50 mM IPA, 100 mM PA, 100 mM L-Asp, 1 mM MgCl₂, 50 μM PLP, 100 mM Tris-HCl, 100 mM potassium phosphate buffer, pH 8.0), and reacted at 25° C. for 2 hours. After the termination of the reaction, the formed 2S,4R-Monatin was quantified, and its concentration was 5.0 mM.

(3) Synthesis of 2S,4R-Monatin from L-Trp

A reaction mixture was prepared so as to contain 5 mg of 1616AT-His (the 1616AT-His solution in Example 5 was used), 0.2 mg of SpAld, 0.4 mL of the cultured medium (TB medium) of pTB2 strain (WO2009/028338) in the Sakaguchi flask, 200 U of superoxide dismutase (Sigma, S8160) and 10 U of oxaloacetate decarboxylase (Sigma, O4878) in 1.0 mL of a reaction solution (50 mM L-Trp, 100 mM PA, 400 mM L-Asp, 1 mM MgCl₂, 50 μM PLP, 100 mM Tris-HCl, 100 mM potassium phosphate buffer, pH 6.5), and reacted at 25° C. for 12 hours. The reaction was performed using a test tube with shaking at 140 rpm. After the termination of the reaction, the formed 2S,4R-Monatin was quantified, and its concentration was 22 mM (44% of yield).

SpAld was prepared by the following method.

A DNA fragment including a SpAld gene was amplified by PCR using plasmid DNA, ptrpSpALD described in Example 5 in JP 2006-204285-A as the template. The primer SpAld-f-NdeI (5′-GGAATTCCATATGACCCAGACGCGCCTCAA-3′: SEQ ID NO:12) and the primer SpAld-r-HindIII (5′-GCCCAAGCTTTCAGTACCCCGCCAGTTCGC-3′: SEQ ID NO:13) were used. E. coli rare codons (6L-ctc, 13L-ctc, 18P-ccc, 38P-ccc, 50P-ccc, 77P-ccc, 81P-ccc and 84R-cga) in an aldolase gene were converted to 6L-ctg, 13L-ctg, 18P-ccg, 38P-ccg, 50P-ccg, 77P-ccg, 81P-ccg and 84R-cgc, respectively. When 6L was converted, the primer 6L-f (5′-ACCCAGACGCGCCTGAACGGCATCATCCG-3′: SEQ ID NO:14) and the primer 6L-r (5′-CGGATGATGCCGTTCAGGCGCGTCTGGGT-3′: SEQ ID NO:15) were used. When 13L was converted, the primer 13L-f (5′-ATCATCCGCGCTCTGGAAGCCGGCAAGCC-3′: SEQ ID NO:16) and the primer 13L-r (5′-GGCTTGCCGGCTTCCAGAGCGCGGATGAT-3′: SEQ ID NO:17) were used. When 18P was converted, the primer 18P-f (5′-GAAGCCGGCAAGCCGGCTTTCACCTGCTT-3′: SEQ ID NO:18) and the primer 18P-r (5′-AAGCAGGTGAAAGCCGGCTTGCCGGCTTC-3′: SEQ ID NO:19) were used. When 38P was converted, the primer 38P-f (5′-CTGACCGATGCCCCGTATGACGGCGTGGT-3′: SEQ ID NO:20) and the primer 38P-r (5′-ACCACGCCGTCATACGGGGCATCGGTCAG-3′: SEQ ID NO:21) were used. When 50P was converted, the primer 50P-f (5′-ATGGAGCACAACCCGTACGATGTCGCGGC-3′: SEQ ID NO:22) and the primer 50P-r (5′-GCCGCGACATCGTACGGGTTGTGCTCCAT-3′: SEQ ID NO:23) were used. When 77P, 81P and 84P were converted, the primer 77P-81P-84R-f (5′-CGGTCGCGCCGTCGGTCACCCCGATCGCGCGCATCCCGGCCA-3′: SEQ ID NO:24) and the primer 77P-81P-84R-r (5′-TGGCCGGGATGCGCGCGATCGGGGTGACCGACGGCGCGACCG-3′: SEQ ID NO:25) were used. PCR was performed using KOD-plus (Toyobo) under the following condition.

| | |
|---|---|
| 1 cycle | 94° C., 2 min |
| 25 cycles | 94° C., 15 sec |
| | 55° C., 15 sec |
| | 68° C., 60 sec |
| 1 cycle | 68° C., 60 sec |
| | 4° C. |

The resulting DNA fragment of about 900 bp was treated with the restriction enzymes NdeI and HindIII, and ligated to pSFN Sm_Aet (Examples 1, 6 and 12 in International Publication WO2006/075486) likewise treated with NdeI and HindIII. E. coli JM109 was transformed with this solution containing the ligated product. The objective plasmid was extracted from ampicillin resistant strains, and this plasmid was designated as pSFN-SpAld.

One loopful of E. coli JM 109/pSFN-SpAld that was the bacterial strain carrying the constructed plasmid pSFN-SpAld was inoculated to 50 mL of LB liquid medium containing 100 mg/L of ampicillin in a 500 mL Sakaguchi flask, and cultured with shaking at 36° C. for 8 hours. After the termination of the culture, 0.0006 mL of the obtained cultured medium was added to 300 mL of a seed liquid medium (10 g of glucose, 5 g of ammonium sulfate, 1.4 g of potassium dihydrogen phosphate, 0.45 g of hydrolyzed soybeans as a nitrogen amount, 1 g of magnesium sulfate heptahydrate, 0.02 g of iron (II) sulfate heptahydrate, 0.02 g of manganese (II) sulfate pentahydrate, 1 mg of thiamin hydrochloride, 0.1 mL of antifoam GD-113K (NOF Corporation), pH 6.3, made to one liter with water) containing 100 mg/L of ampicillin in a 1000 mL volume of jar fermenter, and seed cultivation was started. The seed cultivation was performed at 33° C. with ventilation at 1/1 vvm with stirring at 700 rpm and controlling pH at 6.3 with ammonia until glucose was consumed. Then, 15 mL of the cultured medium obtained as above was added to 285 mL of a main liquid medium (15 g of glucose, 5 g of ammonium sulfate, 3.5 g of phosphoric acid, 0.45 g of hydrolyzed soybeans as the nitrogen amount, 1 g of magnesium sulfate heptahydrate, 0.05 g of iron (II) sulfate heptahydrate, 0.05 g of manganese (II) sulfate pentahydrate, 1 mg of thiamin hydrochloride, 0.1 mL of antifoam GD-113K (NOF Corporation), pH 6.3, made to 0.95 L with water) containing 100 mg/L of ampicillin in a 1000 mL volume of jar fermenter, and main cultivation was started. The main cultivation was performed at 36° C. with ventilation at 1/1 vvm, pH was controlled to 6.3 with ammonia, and stirring was controlled at 700 rpm or more so that the concentration of dissolved oxygen was 5% or more. After glucose contained in the main medium was consumed, the cultivation was continued with dropping a glucose solution at 500 g/L for total 50 hours.

Microbial cells were collected by centrifugation from 100 mL of the obtained cultured medium, washed with and suspended in 20 mM Tris-HCl (pH 7.6), and disrupted by sonication at 4° C. for 30 minutes. Microbial cell debris was removed from the disrupted solution by centrifugation, and the obtained supernatant was used as a soluble fraction.

The above soluble fraction was applied to the anion exchange chromatography column HiLoad 26/10 Q Sepharose HP (supplied from GE health Care Bioscience, CV=53 mL) equilibrated with 20 mM Tris-HCl (pH 7.6), and adsorbed to the carrier. The proteins that had not been adsorbed to the carrier (unadsorbed proteins) were washed out with 20 mM Tris-HCl (pH 7.6), and subsequently, the adsorbed proteins were eluted by linearly changing the concentration of NaCl from 0 mM to 500 mM at a flow rate of 8 mL/minute. Fractions having an aldolase activity were combined, and ammonium sulfate and Tris-HCl (pH 7.6) were added thereto at final concentrations of 1 M and 20 mM, respectively.

The resulting solution was applied to the hydrophobic chromatography column HiLoad 16/10 Phenyl Sepharose HP (supplied from GE health Care Bioscience, CV=20 mL) equilibrated with 1 M ammonium sulfate, 20 mM Tris-HCl (pH 7.6), and adsorbed to the carrier. The proteins that had not been adsorbed to the carrier were washed out with 1 M ammonium sulfate, 20 mM Tris-HCl (pH 7.6), and subsequently, the adsorbed proteins were eluted by linearly changing the concentration of ammonium sulfate from 1 M to 0 M at a flow rate of 3 mL/minute. The fractions having the aldolase activity were combined and concentrated using Amicon Ultra-15 10K (Millipore). The obtained concentrated solution was diluted with 20 mM Tris-HCl (pH 7.6), and used as a SpAld solution. The aldolase activity was measured as an aldol degradation activity using PHOG as the substrate under the following condition.

Reaction condition: 50 mM Phosphate buffer (pH 7.0), 2 mM PHOG, 0.25 mM NADH, 1 mM MgCl₂, 16 U/mL lactate dehydrogenase, an absorbance at 340 nm was measured at 25° C.

pTB2 strain was prepared by the following method.

One loopful of pTB2 strain described in Example 2 in International Publication WO2009/028338 was inoculated to 50 mL of the TB liquid medium containing 100 mg/L of ampicillin in a 500 mL Sakaguchi flask, and cultured with shaking at 37° C. for 16 hours. The obtained cultured medium was used as the cultured medium of pTB2 strain in the Sakaguchi flask (TB medium).

### Example 7

### Synthesis of 2S,4R-Monatin by Microorganisms Having 2S,4R-Monatin Forming Activity

### (1) Synthesis of 2S,4R-Monatin by Bacteria

Rhizobium sp. LAT1, Pseudomonas umorosa AJ11568, Pseudomonas tabaci AJ2778, Xanthomonas oryzae AJ3447, Stenotrophomonas sp. AJ13127, Pseudomonas chlororaphis subsp. chlororaphis NBRC3904, Micrococcus luteus NBRC3067, Xanthomonas albilineans AJ11634, Pseudomonas putida NBRC12668, Pseudomonas betainovorans AJ3735, Pseudomonas putrefaciens AJ1591, Pseudomonas peptidolytica AJ3839, Pseudomonas hydrogenovora AJ3958, Pseudomonas citronocllolis ATCC13674, Arthrobacter ureafaciens AJ1436, Alcaligenes faecalis AJ12469, Rhizobium radiobacter AJ2777, Pseudomonas multivorans AJ3084, Microbacterium sp. AJ2787, Pseudomonas taetrolens ATCC4683, Rhizobium radiobacter ATCC4452, Alcaligenes metalcaligenes AJ2557, Achromobacter brunificans AJ3230, Rhizobium radiobacter NBRC12667, Pseudomonas fragi NBRC3458, Rhizobium radiobacter NBRC12664, Corynebacterium ammoniagenes NBRC12072, Pseudomonas ovalis AJ1594, Rhizobium radiobacter ATCC6466, Pseudomonas synxantha NBRC3912, Rhizobium radiobacter ATCC4720 or Achromobacter butyri AJ2438 was applied onto a nutrient broth (NB) agar medium or the CM2G agar medium (10 g/L of yeast extract, 10 g/L of polypeptone, 5 g/L of glucose, 5 g/L of NaCl, 15 g/L of agar, pH 7.0), and cultured at 30° C. for 2 days.

One loopful of the obtained microbial cells was inoculated to 3 mL of an enzyme production medium (10 g/L of yeast extract, 10 g/L of polypeptone, 1 g/L of glucose, 3 g/L of dipotassium hydrogen phosphate, 1 g/L of potassium dihydrogen phosphate, 0.1 g/L of magnesium sulfate heptahydrate, 5 g/L of ammonium sulfate) in a test tube, which was then cultured with shaking at 30° C. for 16 hours. The microbial cells were collected from 2 mL of the cultured medium by centrifugation, washed with and suspended in 20 mM Tris-HCl (pH 7.6) to prepare 1 mL of a microbial cell suspension.

Then, 1 g of glass beads (0.1 mm) was added to 1 mL of this microbial cell suspension, and the microbial cells were disrupted using the multi beads shocker (Yasui Kikai Co., Ltd.). The resulting disrupted cell solution was centrifuged to use a supernatant as a microbial cell extract.

The reaction of synthesizing 2S,4R-Monatin and the quantification of 2S,4R-Monatin were performed in the same manner as in Example 1, and amounts of the 2S,4R-Monatin which was formed were as follows (Table 2)

TABLE 2
Amount of 2S,4R-Monatin which was formed

| | Amount of |
|---|---|
| | 2S4R-Monatin |
| Microorganism | which was formed |
| Rhizobium sp. LAT1 | 3.8 mM |
| Pseudomonas umorosa AJ11568 | 3.5 mM |
| Pseudomonas tabaci AJ2778 | 3.2 mM |
| Xanthomonas oryzae AJ3447 | 2.7 mM |
| Stenotrophomonas sp. AJ13127 | 2.7 mM |
| Pseudomonas chlororaphis subsp. | 2.6 mM |
| chlororaphis NBRC3904 | |
| Micrococcus luteus NBRC3067 | 2.3 mM |
| Xanthomonas albilineans AJ11634 | 2.2 mM |
| Pseudomonas putida NBRC12668 | 2.2 mM |
| Pseudomonas betainovorans AJ3735 | 2.2 mM |
| Pseudomonas putrefaciens AJ1591 | 2.1 mM |
| Pseudomonas peptidolytica AJ3839 | 2.1 mM |
| Pseudomonas hydrogenovora AJ3958 | 2.0 mM |
| Pseudomonas citronocllolis ATCC13674 | 1.9 mM |
| Arthrobacter ureafaciens AJ1436 | 1.7 mM |
| Alcaligenes faecalis AJ12469 | 1.6 mM |
| Rhizobium radiobacter AJ2777 | 1.5 mM |
| Pseudomonas multivorans AJ3084 | 1.5 mM |
| Microbacterium sp. AJ2787 | 1.5 mM |
| Pseudomonas taetrolens ATCC4683 | 1.4 mM |
| Rhizobium radiobacter ATCC4452 | 1.4 mM |
| Alcaligenes metalcaligenes AJ2557 | 1.4 mM |
| Achromobacter brunificans AJ3230 | 1.4 mM |
| Rhizobium radiobacter NBRC12667 | 1.3 mM |
| Pseudomonas fragi NBRC3458 | 1.3 mM |
| Rhizobium radiobacter NBRC12664 | 1.3 mM |
| Corynebacterium ammoniagenes NBRC12072 | 1.2 mM |
| Pseudomonas ovalis AJ1594 | 1.2 mM |
| Rhizobium radiobacter ATCC6466 | 1.2 mM |
| Pseudomonas synxantha NBRC3912 | 1.1 mM |
| Rhizobium radiobacter ATCC4720 | 1.1 mM |
| Achromobacter butyri AJ2438 | 1.0 mM |

### Synthesis of 2S,4R-Monatin by Actinomycete

Nocardia globerula ATCC21022 was applied onto a YMPG agar medium (3 g/L of yeast extract, 3 g/L of malt extract, 5 g/L of polypeptone, 10 g/L of glucose, 15 g/L of agar, pH 7.0), and cultured at 30° C. for 2 days.

One loopful of the obtained microbial cells was inoculated to 3 mL of a YMPG medium (3 g/L of yeast extract, 3 g/L of malt extract, 5 g/L of polypeptone, 10 g/L of glucose, pH 7.0) in a test tube, and cultured with shaking at 30° C. for 16 hours. The microbial cells were collected from 2 mL of the cultured medium by centrifugation, washed with and suspended in 20 mM Tris-HCl (pH 7.6) to prepare 1 mL of a microbial cell suspension.

Then, 1 g of glass beads (0.1 mm) was added to 1 mL of this microbial cell suspension, and the microbial cells were disrupted using the multi beads shocker (Yasui Kikai Co., Ltd.). The resulting disrupted cell solution was centrifuged to use a supernatant as a microbial cell extract.

The reaction of synthesizing 2S,4R-Monatin and the quantification of 2S,4R-Monatin were performed in the same manner as in Example 1, and amount of the 2S,4R-Monatin which was formed was as follows (Table 3)

TABLE 3
Amount of 2S,4R-Monatin which was formed

| | Amount of 2S4R-Monatin |
|---|---|
| Microoganism | which was formed |
| Nocardia globerula ATCC21022 | 0.57 mM |

(3) Synthesis of 2S,4R-Monatin by Yeast

Lodderomyces elongisporus CBS2605, Candida norvegensis NBRC0970, Candida inconspicua NBRC0621 or Yarrowia lypolytica NBRC0746 was applied onto a YPD agar medium (10 g/L of yeast extract, 20 g/L of polypeptone, 20 g/L of glucose, 15 g/L of agar), and cultured at 30° C. for 2 days.

One loopful of the obtained microbial cells was inoculated to 3 mL of a YPD medium (10 g/L of yeast extract, 20 g/L of polypeptone, 20 g/L of glucose) in a test tube, and cultured with shaking at 30° C. for 16 hours. The microbial cells were collected from 2 mL of the cultured medium by centrifugation, washed with and suspended in 20 mM Tris-HCl (pH 7.6) to prepare 1 mL of a microbial cell suspension.

Then, 1 g of glass beads (0.5 mm) was added to 1 mL of this microbial cell suspension, and the microbial cells were disrupted using the multi beads shocker (Yasui Kikai Co., Ltd.). The resulting disrupted cell solution was centrifuged to use a supernatant as a microbial cell extract.

The reaction of synthesizing 2S,4R-Monatin and the quantification of 2S,4R-Monatin were performed in the same manner as in Example 1, and amount of the 2S,4R-Monatin which was formed were as follows (Table 4)

TABLE 4
Amount of 2S,4R-Monatin which was formed

| | |
|---|---|
| | Amount of 2S4R-Monatin |
| Microorganism | which was formed |

| | |
|---|---|
| Lodderomyces elongisporus CBS2605 | 0.57 mM |
| Candida norvegensis NBRC0970 | 0.55 mM |
| Candida inconspicua NBRC0621 | 0.52 mM |
| Yarrowia lypolytica NBRC0746 | 0.52 mM |

### Example 8

### Production of 2S,4R-Monatin Potassium Salt Dihydrate

After 149.00 g of ethanol was added to a reduction reaction concentrated solution (containing 36.62 g (125.28 mmol) of Monatin, (2S, 4R):(2R,4R)=32:68), 0.25 g of 2R,4R-Monatin potassium salt monohydrate was added as a seed crystal, and the mixture was stirred at 56° C. for 4 hours to perform preferential crystallization of the 2R,4R-Monatin potassium salt monohydrate. The crystallized crystal was separated by filtration (wet crystal 31.27 g) to obtain 225.80 g of a mother solution (containing 22.41 g (76.68 mmol) of Monatin, (2S, 4R):(2R,4R)=53:47). This mother solution was cooled to 10° C. and stirred for 5 hours to crystallize 2S,4R-Monatin potassium salt dihydrate. The crystal was separated by filtration (wet crystal 32.74 g), and dried under reduced pressure to yield 9.88 g (15.68 mmol) of the objective 2S,4R-Monatin potassium salt dihydrate (HPLC purity: 55.5%). Then, 9.35 g of this crude crystal was dissolved in 25.37 g of water, and 58.99 g of ethanol was added to this dissolved solution, which was stirred at 25° C. for 5 hours to perform delicate crystallization of the 2S,4R-Monatin potassium salt dihydrate. The crystal was separated by filtration (wet crystal 4.49 g), and dried under reduced pressure to yield 3.75 g (9.62 mmol) of the objective 2S,4R-Monatin potassium salt dihydrate (HPLC purity: 96.0%).

A water content and a potassium content of the obtained crystal (2S,4R-Monatin potassium salt dihydrate) were analyzed by a water measurement method and a cation analysis method using ion chromatography. Details of the performed water measurement method and cation analysis method are shown below.

(Water Measurement Method)

Measurement apparatus: Hiranuma Automatic Water Measurement

Apparatus AQV-2000 (supplied from Hiranuma Sangyo Corporation)
Measurement condition: Titration solution=Hydranal
Composite 5K (supplied from Riedel de Haen)

(Cation Analysis Method)

### Apparatus: Tosoh 102001

### Column: TSKgel SuperIC-Cation (4.6×150 mm)

Guard column: TSKgel SuperIC-Cation (1 cm)

### Suppress gel: TSKgel TSKsuppressIC-C

Column temperature: 40° C.
Eluant flow: 0.7 mL/minute
Sample injection amount: 30 μL
Detection: Electric conductivity
Eluant composition: 2.2 mM methanesulfonic acid+1.0 mM 18-crown-6-ether+0.5 mM histidine mixed aqueous solution

¹HNMR (400 MHz, D₂O) δ: 2.11 (dd, J=19.0, 27.0 Hz, 1H), 2.39 (dd, J=5.0, 27.0 Hz, 1H), 3.14 (s, 2H), 3.90 (dd, J=5.0, 19.0 Hz, 1H), 7.06 (m, 1H), 7.13 (m, 1H), 7.15 (s, 1H), 7.40 (d, 8.5 Hz, 1H), 7.6 (d, 8.5 Hz, 1H)

ESI-MS Calculated value: C₁₄H₁₆N₂O₅=292.11

ESI-MS Analyzed value: C₁₄H₁₆N₂O₅=290.9 M-H]⁻

### Example 9

### Isomerization Reaction Using 5-nitrosalicylaldehyde

0.15 g (0.38 mmol) of the 2S,4R-Monatin potassium salt dihydrate obtained in Example 8 was added to 10.0 g of an aqueous solution of 70% ethanol, and completely dissolved at 60° C. 7.6 mg (0.045 mmol) of 5-nitrosalicylaldehyde and 7.5 μL (0.13 mmol) of acetic acid were added to that dissolved solution, and stirred at 60° C. for 48 hours. The reaction solution was analyzed and quantified by HPLC, and a molar ratio of 2S,4R-Monatin and 2R,4R-Monatin in the reaction solution was 1:2.1.

### Example 10

### Isomerization Reaction Using Pyridoxal Hydrochloride Salt

0.15 g (0.38 mmol) of the 2S,4R-Monatin potassium salt dihydrate obtained in Example 8 was added to 10.0 g of the aqueous solution of 70% ethanol, and completely dissolved at 60° C. 9.1 mg (0.045 mmol) of pyridoxal hydrochloride and 7.5 μL (0.13 mmol) of acetic acid were added to that dissolved solution, and stirred at 60° C. for 48 hours. The reaction solution was analyzed and quantified by HPLC, and the molar ratio of 2S,4R-Monatin and 2R,4R-Monatin in the reaction solution was 1:1.3.

### Example 11

### Isomerization Reaction Using Pyridoxal 5-Phosphate Monohydrate

0.15 g (0.38 mmol) of the 2S,4R-Monatin potassium salt dihydrate obtained in Example 8 was added to 10.0 g of the aqueous solution of 70% ethanol, and completely dissolved at 60° C. 12.8 mg (0.048 mmol) of pyridoxal 5-phosphate monohydrate and 7.5 μL (0.13 mmol) of acetic acid were added to that dissolved solution, and stirred at 60° C. for 48 hours. The reaction solution was analyzed and quantified by HPLC, and the molar ratio of 2S,4R-Monatin and 2R,4R-Monatin in the reaction solution was 1:1.1.

### Example 12

### Isomerization Reaction Using Salicylaldehyde

0.15 g (0.38 mmol) of the 2S,4R-Monatin potassium salt dihydrate obtained in Example 8 was added to 10.0 g of the aqueous solution of 70% ethanol, and completely dissolved at 60° C. 5.3 mg (4.6 μL, 0.043 mmol) of salicylaldehyde and 7.5 μL (0.13 mmol) of acetic acid were added to that dissolved solution, and stirred at 60° C. for 48 hours. The reaction solution was analyzed and quantified by HPLC, and the molar ratio of 2S,4R-Monatin and 2R,4R-Monatin in the reaction solution was 1:0.6.

### Example 13

### Isomerization Reaction Using 3,5-Dichlorosalicylaldehyde

0.15 g (0.38 mmol) of the 2S,4R-Monatin potassium salt dihydrate obtained in Example 8 was added to 10.0 g of the aqueous solution of 70% ethanol, and completely dissolved at 60° C. 8.1 mg (0.042 mmol) of 3,5-dichlorosalicylaldehyde and 7.5 μL (0.13 mmol) of acetic acid were added to that dissolved solution, and stirred at 60° C. for 48 hours. The reaction solution was analyzed and quantified by HPLC, and the molar ratio of 2S,4R-Monatin and 2R,4R-Monatin in the reaction solution was 1:1.5.

### Example 14

### Production of 2R,4R-Monatin Potassium Salt Monohydrate by Isomerization-Crystallization Using 2S,4R-Monatin Potassium Salt Dihydrate as Starting Material

The 2S,4R-Monatin potassium salt dihydrate is added to an aqueous solution of 20% ethanol and completely dissolved at 60° C. 5 molar percent 5-Nitrosalicylaldehyde relative to the 2S,4R-Monatin, and 30 molar percent acetic acid relative to the 2S,4R-Monatin are added to this dissolved solution, and stirred for 48 hours. Ethanol at a final concentration of 70% is added to this reaction solution (2S,4R-Monatin:2R,4R-Monatin=1:2.1), subsequently one percent 2R,4R-Monatin potassium salt monohydrate relative to the 2R,4R-Minatin in the reaction solution is added as the seed crystal thereto, and the mixture is stirred at 60° C. for 48 hours to perform the isomerization-crystallization. The crystallized crystal is separated by filtration, and dried under reduced pressure to yield the objective 2R,4R-Monatin potassium salt monohydrate.

### Example 15

### Isomerization Reaction Using Glyoxylic Acid

0.15 g (0.38 mmol) of the 2S,4R-Monatin potassium salt dihydrate obtained in Example 1 was added to 10.0 g of the aqueous solution of 70% ethanol, and completely dissolved at 60° C. 5.1 mg (0.069 mmol) of glyoxylic acid and 7.5 μL (0.13 mmol) of acetic acid were added to that dissolved solution, and stirred at 60° C. for 48 hours. The reaction solution was analyzed and quantified by HPLC, and the molar ratio of 2S,4R-Monatin and 2R,4R-Monatin in the reaction solution was 1:0.07

### INDUSTRIAL APPLICABILITY

As described above, the methods of the present invention are useful for producing the Monatin which can be used as the sweetener.

### SEQUENCE LISTING FREE TEXT

SEQ ID NO:1: Nucleotide sequence of aminotransferase gene derived from Bacillus sp.

SEQ ID NO:2: Amino acid sequence of aminotransferase derived from Bacillus sp.

SEQ ID NO:3: Nucleotide sequence of aminotransferase gene (nucleotide numbers 231-1538) and the upstream and downstream regions thereof which are derived from Bacillus sp.

SEQ ID NO:4: Amino acid sequence of a fragment of aminotransferase derived from Bacillus sp.

SEQ ID NO:5: Amino acid sequence of a fragment of aminotransferase derived from Bacillus sp.

SEQ ID NO:6: Forward primer for amplifying DNA fragment containing aminotransferase gene derived from Bacillus sp. (Bp-u200-f)

SEQ ID NO:7: Reverse primer for amplifying DNA fragment containing aminotransferase gene derived from Bacillus sp. (Bp-d200-r)

SEQ ID NO:8: Forward primer for amplifying DNA fragment containing aminotransferase gene derived from Bacillus sp. (1616AT-Nde-f)

SEQ ID NO:9: Reverse primer for amplifying DNA fragment containing aminotransferase gene derived from Bacillus sp. (1616-xho-r)

SEQ ID NO:10: Forward primer for converting DNA sequence recognized by NdeI, which is found on aminotransferase gene derived from Bacillus sp. (1616-delNde-f)

SEQ ID NO:11: Reverse primer for converting DNA sequence recognized by NdeI, which is found on aminotransferase gene derived from Bacillus sp. (1616-delNde-r)

SEQ ID NO:12: Forward primer for amplifying DNA fragment containing SpAld gene (SpAld-f-NdeI)

SEQ ID NO:13: Reverse primer for amplifying DNA fragment containing SpAld gene (SpAld-r-HindIII)

SEQ ID NO:14: Forward primer for converting rare codon 6L in SpAld gene (6L-f)

SEQ ID NO:15: Reverse primer for converting rare codon 6L in SpAld gene (6L-r)

SEQ ID NO:16: Forward primer for converting rare codon 13L in SpAld gene (13L-f)

SEQ ID NO:17: Reverse primer for converting rare codon 13L in SpAld gene (13L-r)

SEQ ID NO:18: Forward primer for converting rare codon 18P in SpAld gene (18P-f)

SEQ ID NO:19: Reverse primer for converting rare codon 18P in SpAld gene (18P-r)

SEQ ID NO:20: Forward primer for converting rare codon 38P in SpAld gene (38β-f)

SEQ ID NO:21: Reverse primer for converting rare codon 38P in SpAld gene (38P-r)

SEQ ID NO:22: Forward primer for converting rare codon 502 in SpAld gene (50β-f)

SEQ ID NO:23: Reverse primer for converting rare codon 50P in SpAld gene (50P-r)

SEQ ID NO:24: Forward primer for converting rare codons 77P, 81P and 84R in SpAld gene (77P-81P-84R-f)

SEQ ID NO:25: Reverse primer for converting rare codons 77P, 81P and 84R in SpAld gene (77P-81P-84R-r)

1. A method for producing 2S,4R-Monatin or a salt thereof, comprising contacting 4R-IHOG with an L-aminotransferase in the presence of an L-amino acid to form the 2S,4R-Monatin.

2. The production method of claim 1, further comprising contacting an oxy derivative of the L-amino acid with a decarboxylase to degrade the oxy derivative of the L-amino acid, wherein the oxy derivative of the L-amino acid is formed from the L-amino acid due to action of the L-aminotransferase.

3. The production method of claim 1 or 2, wherein the L-amino acid is L-aspartate.

4. The production method of claim 3, further comprising contacting oxaloacetate with an oxaloacetate decarboxylase to irreversibly form pyruvate, wherein the oxaloacetate is formed from the L-aspartate by action of the L-aminotransferase.

5. The production method of any one of claims 1-4, wherein the L-aminotransferase is derived from a microorganism belonging to genus Achromobacter, genus Alcaligenes, genus Arthrobacter, genus Bacillus, genus Candida, genus Corynebacterium, genus Lodderomyce, genus Micrococcus, genus Microbacterium, genus Nocardia, genus Pseudomonas, genus Rhizobium, genus Stenotrophomonas, genus Xanthomonas, or genus Yarrowia.

6. The production method of claim 5, wherein the L-aminotransferase is derived from a microorganism belonging to Achromobacter brunificans, Achromobacter butyri, Alcaligenes faecalis, Alcaligenes metalcaligenes, Arthrobacter ureafaciens, Bacillus sp., Candida norvegensis, Candida inconspicua, Corynebacterium ammoniagenes, Lodderomyces elongisporus, Micrococcus luteus, Microbacterium sp., Nocardia globerula, Pseudomonas betainovorans, Pseudomonas chlororaphis, Pseudomonas citronocllolis, Pseudomonas fragi, Pseudomonas hydrogenovora, Pseudomonas multivorans, Pseudomonas ovalis, Pseudomonas peptidolytica, Pseudomonas putida, Pseudomonas putrefaciens, Pseudomonas synxantha, Pseudomonas tabaci, Pseudomonas taetrolens, Pseudomonas umorosa, Rhizobium radiobacter, Rhizobium sp., Stenotrophomonas sp., Xanthomonas albilineans, Xanthomonas oryzae, or Yarrowia lypolytica.

7. The production method of any one of claims 1-4, wherein the L-aminotransferase consists of an amino acid sequence showing 90% or more identity to the amino acid sequence represented by SEQ ID NO:2.

8. The production method of any one of claims 1-7, wherein the 4R-IHOG is contacted with the L-aminotransferase using a transformant that expresses the L-aminotransferase.

9. The production method of any one of claims 1-8, further comprising condensing indole-3-pyruvate and pyruvate to form the 4R-IHOG.

10. The production method of claim 9, the indole-3-pyruvate and the pyruvate are condensed by contacting the indole-3-pyruvate and the pyruvate with an aldolase.

11. The production method of claim 9 or 10, wherein at least part of the pyruvate used in the formation of the 4R-IHOG is from pyruvate formed from the oxaloacetate due to action of the oxaloacetate decarboxylase.

12. The production method of any one of claims 9-11, further comprising oxidizing a tryptophan to form the indole-3-pyruvate.

13. The production method of claim 12, wherein the tryptophan is oxidized by contacting the tryptophan with a deamination enzyme.

14. The production method of any one of claims 9-13, wherein the production of the 2S,4R-Monatin or the salt thereof is carried out in one reactor.

15. A method for producing 2R,4R-Monatin or a salt thereof, comprising the following (I) and (II):
(I) performing the method of any one of claims 1-14 to form the 2S,4R-Monatin; and
(II) isomerizing the 2S,4R-Monatin to form the 2R,4R-Monatin.

16. The production method of claim 15, wherein the 2S,4R-Monatin is isomerized in the presence of an aromatic aldehyde.

17. An L-aminotransferase that is a protein selected form the group consisting of the following (A)-(D):
(A) a protein consisting of the amino acid sequence represented by SEQ ID NO:2;
(B) a protein comprising the amino acid sequence represented by SEW ID NO:2;
(C) a protein consisting of an amino acid sequence showing 90% or more identity to the amino acid sequence represented by SEQ ID NO:2, and having an L-aminotransferase activity; and
(D) a protein consisting of an amino acid sequence comprising mutation of one or several amino acid residues, which is selected from the group consisting of deletion, substitution, addition and insertion of the amino acid residues in the amino acid sequence represented by SEQ ID NO:2, and having an L-aminotransferase activity.

18. A polynucleotide selected from the group consisting of the following (a)-(e):
(a) a polynucleotide consisting of the nucleotide sequence represented by SEQ ID NO:1;
(b) a polynucleotide comprising the nucleotide sequence represented by SEQ ID NO:1;
(c) a polynucleotide consisting of a nucleotide sequence showing 90% or more identity to the amino acid sequence represented by SEQ ID NO:1, and encoding a protein having an L-aminotransferase activity;
(d) a polynucleotide that hybridizes under a stringent condition with a polynucleotide consisting of the nucleotide sequence complementary to the nucleotide sequence represented by SEQ ID NO:1, and encodes a protein having an L-aminotransferase activity; and
(e) a polynucleotide encoding the protein of claim 17.

19. An expression vector comprising the polynucleotide of claim 18.

20. A transformant introduced with the expression vector of claim 19.

21. A method for producing an L-amino transfearase, comprising culturing the transformant of claim 20 in a medium to obtain the L-aminotransferase.

22. A method of producing 2S,4R-Monatin or a salt thereof, comprising contacting 4R-IHOG with the L-aminotransferase of claim 17 in the presence of an L-amino acid to form the 2S,4R-Monatin.

23. A method for producing 2R,4R-Monatin or a salt thereof, comprising the following (I′) and (II′):
(I′) performing the method of claim 22 to form the 2S,4R-Monatin; and
(II′) isomerizing the 2S,4R-Monatin to form the 2R,4R-Monatin.

24. The production method of claim 23, wherein the 2S,4R-Monatin is isomerized in the presence of an aromatic aldehyde.
